(19) United States
(12) Patent Application Publication
(10) Pub. No.: US 2011/0274713 A1
(43) Pub. Date: Nov. 10, 2011

(54) ANTIGEN-PRESENTING SCAFFOLDS

(75) Inventors: Paul Leslie Burn, Queensland (AU); Joanna T. Blanchfield, Queensland (AU); George Vamvounis, Brisbane (AU); Yogendra Singh, Queensland (AU); Melissa Lee Foster, Plainland (AU)

(73) Assignee: The University of Queensland, St. Lucia (AU)

(21) Appl. No.: 13/057,677

(22) PCT Filed: Aug. 4, 2009
(86) PCT No.: PCT/AU09/00989

§ 371 Date: Jul. 22, 2011

Related U.S. Application Data
(60) Provisional application No. 61/086,452, filed on Aug. 5, 2008.

(51) Int. Cl. A61K 39/385; A61P 37/00; C07K 17/06

(52) U.S. Cl. 424/194.1; 530/323

(57) ABSTRACT

The invention relates to compounds having formula (I): Scaffold-[L-(Antigen)ₜ]_{y} (I) wherein Antigen represents at least a portion of a target antigen for modulating an immune response; wherein t is 0 or an integer of at least 1; wherein y is at least 1; wherein the number of Antigens on the Scaffold is at least 2; wherein L is a linking group or a covalent bond, wherein when L is a covalent bond, the covalent bond is a single bond attached to a sp or sp² hybridized atom of the Scaffold and when L is a linking group, the linking group is attached to the Scaffold through a single bond attached to a sp or sp² hybridized atom; whereby the Scaffold is sufficiently rigid to maintain the relative position of the single bonds attached to sp or sp² hybridized atoms. Further described are compositions containing the compounds and methods of using them.

### FIELD OF THE INVENTION

This invention relates generally to immunomodulating compositions. More particularly, the present invention relates to antigen-presenting scaffolds having a rigid structure for presenting antigens to the immune system and to methods of making and using such scaffolds.

### BACKGROUND

Vaccines that present multiple antigens to the immune system, such as multiple antigenic peptides (MAPs), have been used to improve immune response. For example, multiple peptides may be synthesized or grafted upon a small polylysine branched structures to enhance antigenic properties relative to the individual peptides. However, in many cases, the immune response produced by immunisation with such dendrimers have been disappointing or less than optimal.

MAPs are comprised of flexible arms, which may fold back towards the centre potentially limiting the accessibility of the pendant antigens to the immune system, or alternatively the antigens may be located randomly and variably in space.

Another drawback of MAPs is that they are typically prepared using divergent synthesis, which results in poor control in attaching a finite number of antigens to the structure and in regulating completion of reaction steps leading to a heterogenous population of structures of inconsistent shape.

Dendritic compounds (otherwise known as “dendrimers”) are macromolecules, variously referred to in the literature as hyperbranched dendrimers, arborols, fractal polymers and starburst dendrimers, illustrative examples of which include polyamidoamine (PAMAM), polypropylene imine) dendrimers, poly L-lysine and N,N′-bis(acrylamido)acetic acid dendrimers.

Dendrimers have a central core and attached dendrons, also known as dendrites. Dendrons are branched structures comprising branching units and optionally linking units. The generation of a dendron is defined by the number of levels of branching. Dendrons with the same structure (architecture) but a higher generation, or order, are composed of the same structural units (branching and linking units) but have an additional level of branching. There can be reactive end groups on the periphery or distal units of the dendrons. Dendrimers can be comprised of dendrons with different branching and linking groups and/or generations.

The present invention is predicated in part on the determination that dendrimers such as MAPs have flexible dendrites, which may fold back towards the central core or present the antigens in an unsuitable position and thereby limit accessibility of the pendant antigens to the immune system or be located randomly and variably in space. Based on this determination, the present inventors consider that better immune responses can be generated using rigid scaffold structures as vehicles for presentation of antigen(s) to the immune system maximising the number of antigens presented and providing predictability in where the antigens are being presented in space. The present inventors have also developed a synthetic procedure for preparing rigid scaffolds with improved control of the number and position of antigens presented on the periphery of the scaffold.

### SUMMARY OF THE INVENTION

In one aspect of the present invention there is provided a compound having formula (I):

Scaffold-[L-(Antigen)ₜ]_{y} (I)

wherein Antigen represents at least a portion of a target antigen for modulating an immune response;
wherein t is 0 or an integer of at least 1;
wherein y is at least 1;
wherein the number of Antigens on the Scaffold is at least 2;
wherein L is a linking group or a covalent bond, wherein when L is a covalent bond, the covalent bond is a single bond attached to a sp or sp² hybridized atom of the Scaffold and when L is a linking group, the linking group is attached to the Scaffold through a single bond attached to a sp or sp² hybridized atom; whereby the Scaffold is sufficiently rigid to maintain the relative position of the single bonds attached to sp or sp² hybridized atoms.

In some embodiments, the Scaffold comprises a central acetylenyl moiety or a central cyclic moiety such as an aryl group, caged hydrocarbon group or silsesquioxane group or mixtures thereof. In some embodiments, the Scaffold comprises at least partially conjugated unbranched moiety. In other embodiments, the Scaffold is an at least partially conjugated branched moiety.

In another aspect of the invention there is provided a compound having formula (II):

Scaffold-[L-(Antigen)ₜ]_{y} (II)

wherein the Scaffold comprises a group

Core-[Spacer]_{z}

wherein the Core is a central atom or group and the spacer is a group wherein each Spacer, alone or in combination with the Core, comprises at least one unbranched or branched moiety comprising at least one group selected from aryl, heteroaryl, alkenyl, acetylenyl and carbonyl,
wherein t is 0 or an integer of at least 1;
wherein y is at least 1;
wherein the number of Antigens on the Scaffold is at least 2;
wherein Antigen represents at least a portion of a target antigen for modulating an immune response;
wherein z is at least 1;
wherein L is a linking group or a covalent bond, wherein when L is a covalent bond, the covalent bond is a single bond attached to a sp or sp² hybridized atom of the spacer and when L is a linking group, the linking group is attached to the spacer(s) through a single bond attached to a sp or sp² hybridized atom; whereby the Scaffold is sufficiently rigid to maintain the relative position of the single bonds attached to sp or sp² hybridized atoms.

In yet another aspect of the invention there is provided a compound having formula (III):

Scaffold[L-(Antigen)ₜ]_{y} (III)

wherein the Scaffold comprises a group

Core-[Spacer]_{z}

wherein the Core is a central atom or group and the Spacer is a group wherein each Spacer, alone or in combination with the Core, comprises at least one partially conjugated unbranched or branched moiety comprising at least two groups selected from aryl, heteroaryl, alkenyl, acetylenyl and carbonyl,
wherein t is 0 or an integer of at least 1;
wherein y is at least 1;
wherein the number of Antigens on the Scaffold is at least 2;
wherein Antigen represents at least a portion of a target antigen for modulating an immune response;
wherein z is at least 1;
wherein L is a linking group or a covalent bond, wherein when L is a covalent bond, the covalent bond is a single bond attached to a sp or sp² hybridized atom of the spacer and when L is a linking group, the linking group is attached to the spacer(s) through a single bond attached to a sp or sp² hybridized atom; whereby the Scaffold is sufficiently rigid to maintain the relative position of the single bonds attached to sp or sp² hybridized atoms.

In some embodiments, the Core comprises an acetylenyl moiety or a cyclic moiety such as an aryl group, caged hydrocarbon group or silsesquioxane group or mixtures thereof. In some embodiments, the Spacer is an at least partially conjugated unbranched moiety. In other embodiments, the Spacer is an at least partially conjugated branched moiety. In some embodiments, each Spacer bears one antigen whereas in other embodiments at least one Spacer bears more than one antigen. In some embodiments, each Spacer bears more than one antigen.

In certain embodiments, the Spacer-[L-(Antigen)] may be provided in the form of a DENDRITE.

In one particular embodiment of the invention there is provided a compound having the formula (IV):

CORE-[DENDRITE]n (IV)

wherein CORE represents an atom or group, n represents an integer of at least 1, and DENDRITE, which may be the same or different if n is greater than 1, represents an at least partly conjugated dendritic molecular structure comprising groups selected from aryl, heteroaryl, alkenyl, acetylenyl and carbonyl, and said DENDRITE comprising at least one antigen; and wherein said CORE terminating at a bond to a sp² hybridized atom which forms part of a moiety that has at least two substituents.

Suitably, an individual antigen represents at least a portion of a target antigen to which modulation (e.g., stimulation, enhancement or attenuation) of an immune response is desired. For example, the target antigen can be selected from foreign or endogenous antigens, as described for example below. The antigen and target antigen can be any type of biological molecule including, for example, simple intermediary metabolites, sugars, lipids, and hormones as well as macromolecules such as complex carbohydrates, phospholipids, nucleic acids, polypeptides and peptides.

In another aspect, the present invention provides immunomodulating compositions, which comprise a compound as broadly described above and optionally a pharmaceutically acceptable carrier, diluent or adjuvant.

Yet another aspect of the present invention provides methods for modulating an immune response in a subject. These methods generally comprise administering to the subject a compound as broadly described above, and optionally a pharmaceutically acceptable carrier, diluent or adjuvant. The active components of the composition may be administered sequentially, separately or simultaneously. In some embodiments, the immune response is a B-cell mediated immune response. In other embodiments, the immune response is a T-cell mediated immune response. Advantageously, these methods are useful for treating or preventing a disease or condition associated with the presence or aberrant expression of at least one target antigen in a subject. In some embodiments, the disease or condition is treated or prevented by using a compound as broadly described above, wherein the or each antigen of the compound corresponds to at least a portion of a corresponding target antigen, and stimulates or otherwise enhances an immune response to that target antigen. In these embodiments, the disease or condition is selected from a pathogenic infection, a disease characterised by immunodeficiency or a cancer.

In other embodiments, the disease or condition is treated or prevented by using a compound as broadly described above, wherein the or each antigen of the compound corresponds to at least a portion of a corresponding target antigen, and attenuates or otherwise suppresses or reduces an immune response or elicits a tolerogenic response to that target antigen. In these embodiments, the disease or condition is selected from transplant rejection, graft versus host disease, allergies, parasitic diseases, inflammatory diseases and autoimmune diseases.

In a further aspect, the invention contemplates the use of a compound as broadly defined above for modulating an immune response to a target antigen.

In still another aspect, the invention resides in the use of a compound as broadly defined above in the manufacture of a medicament for treating or preventing a disease or condition associated with the presence or aberrant expression of a target antigen.

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “antigen” is meant all, or part of, a protein, peptide, carbohydrate, or other molecule or macromolecule capable of eliciting an immune response in a vertebrate animal, especially a mammal. Such antigens are also reactive with antibodies from animals immunized with that protein, peptide, saccharide, carbohydrate, or other molecule or macromolecule.

By “alloantigen” is meant an antigen found only in some members of a species, such as blood group antigens. By contrast a “xenoantigen” refers to an antigen that is present in members of one species but not members of another. Correspondingly, an “allograft” is a graft between members of the same species and a “xenograft” is a graft between members of a different species.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

The term “CORE” as used herein refers to an atom or group capable of presenting rigid unbranched or branched spacers including dendrites, in a two or three dimensional structure.

By “corresponds to” or “corresponding to” is meant an antigen which comprises an amino acid sequence that displays substantial similarity to an amino acid sequence in a target antigen. In general the antigen will display at least about 30, 40, 50, 55, 60, 65, 70, 75, 80, 85, 90, 91, 92, 93, 94, 95, 96, 97, 98, 99% similarity to at least a portion of the target antigen. In some embodiments, the term “represents” encompasses amino acid sequences that correspond to an amino acid sequence of at least a portion of a target antigen.

The term “DENDRITE” as used herein refers to a moiety that comprises a branched dendritic structure including groups selected from aryl, heteroaryl, alkenyl, acetylenyl and carbonyl groups and at least one antigen. The branched structure begins with an aryl, heteroaryl or alkenyl moiety having at least two further substituents that provide further components of the DENDRITE in addition to the linkage to the CORE.

By “effective amount,” in the context of modulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., monkeys), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), mustela (e.g., ferrets), avians (e.g., chickens, turkeys, ducks, geese, companion birds such as canaries, budgerigars etc), marine mammals (e.g., dolphins, whales), reptiles (snakes, frogs, lizards etc), and fish. A preferred subject is a human in need of treatment or prophylaxis for a condition or disease, which is associated with the presence or aberrant expression of an antigen of interest. However, it will be understood that the aforementioned terms do not imply that symptoms are present.

By “pharmaceutically-acceptable carrier” is meant a solid or liquid filler, diluent or encapsulating substance that may be safely used in topical or systemic administration.

“Polypeptide,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “suppression,” “suppressing” and the like is meant any attenuation or regulation of an immune response, including B-lymphocyte and T-lymphocyte immune responses, to an antigen or group of antigens. In some embodiments, the attenuation is mediated at least in part by suppressor T-lymphocytes (e.g., CD4⁺CD25⁺ regulatory T-lymphocytes).

By “treatment,” “treat,” “treated” and the like is meant to include both therapeutic and prophylactic treatment.

As used herein, the term “aryl” is intended to mean any stable, monocyclic or polycyclic aromatic carbon containing ring system of up to 7 atoms in each ring, wherein at least one ring is aromatic. Examples of such aryl groups include, but are not limited to, phenyl, naphthyl, indanyl, azulene, anthracene, phenanthrene, phenalene, fluorene, biphenyl and binaphthyl.

As used herein the term “conjugated” refers to moieties having alternating single and multiple bonds allowing electrons to become delocalized within the moiety. In a conjugated system, the p-orbitals on adjacent atoms are aligned such that the electrons are delocalized within the p-orbitals. This alignment of p-orbitals increases “double bond character” of the single bonds in the conjugated system and thereby reduces rotation and flexibility. For example, conjugation may occur where a phenyl ring is directly bonded to another phenyl ring or an ethenyl or acetylenyl group or where two phenyl rings are linked together with an intervening ethenyl or acetylenyl group. A moiety may be fully conjugated where the moiety consists of alternating single and double bonds and aromatic systems and electrons are delocalized within the entire moiety. A moiety may be fully conjugated where the moiety consists of alternating single and double bonds and aromatic systems without the electrons being fully delocalized within the entire moiety because of the linking arrangement. For example, 1,3,5-triphenylbenzene is fully conjugated but the pi-electrons of the phenyl substituents are not delocalized between one another. That is the moiety is at least partially conjugated.

The term “heteroaryl” as used herein, represents a stable monocyclic or polycyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, pyrazolyl, indolyl, benzotriazolyl, furanyl, thienyl, thiophenyl, benzothienyl, benzofuranyl, quinolinyl, isoquinolinyl, oxazolyl, isoxazolyl, imidazolyl, pyrazinyl, pyridazinyl, pyridinyl, pyrimidinyl, pyrrolyl, tetrahydroquinoline, thiazolyl, isothiazolyl, 1,2,4-triazolyl, 1,2,4-oxadiazolyl, 1,2,4-thiadiazolyl, benzodioxanyl, benzazepinyl, benzoxepinyl, benzodiazepinyl, benzothiazepinyl and benzothiepinyl. Preferred heteroaryl groups have 5- or 6-membered rings, such as pyrazolyl, furanyl, thienyl, oxazolyl, isoxazolyl, imidazolyl, pyrazinyl, pyridazinyl, pyridinyl, pyrimidinyl, pyrrolyl, thiazolyl, isothiazolyl, 1,2,3-triazolyl, 1,2,4-triazolyl and 1,2,4-oxadiazolyl and 1,2,4-thiadiazolyl.

The term “alkenyl” as used herein refers to a straight chain or branched unsaturated hydrocarbon group having 2 to 10 carbon atoms and at least one double bond. Where appropriate, the alkenyl group may have a specified number of carbon atoms, for example, C₂₋₆ alkenyl which include alkenyl groups having 2, 3, 4, 5, or 6 carbon atoms in a linear or branched arrangement. Examples of suitable alkenyl groups include, but are not limited to, ethenyl, propenyl, 1-butenyl, 2-butenyl 1,3-butadienyl, 1-pentenyl, 2-pentenyl, 3-pentenyl, 4-pentenyl, 1,3-pentadienyl, 1,4-pentadienyl, 2,4-pentadienyl, 1-hexenyl, 2-hexenyl, 3-hexenyl, 4-hexenyl, 5-hexenyl, 1,3-hexadienyl, 1,4-hexadienyl, 1,5-hexadienyl, 2,4-hexadienyl, 1,3,5-hexatrienyl, heptenyl, octenyl, nonenyl and decenyl.

As used herein the term “acetylenyl” refers to an ethynyl group of the formula: —C≡C—.

The term “caged hydrocarbon” refers to a compound composed of carbon and hydrogen atoms that contains three or more rings arranged topologically so as to enclose a volume of space. The carbon framework of caged hydrocarbons is rigid allowing geometric relationships of substituents on the caged hydrocarbon to be well defined. Caged hydrocarbons include, but are not limited to adamantane, tetrahedrane, cubane, diamantine, triamantane, prismane, dodecahedrane and 2,2,2-bicyclooctane.

The term “caged silicon compound” refers to a compound comprising silicon atoms that contains three or more rings arranged topologically to enclose a volume of space. Caged silicon compounds may also include oxygen atoms in the ring structure. An example of a caged silicon compound is silesquioxane.

As used herein, the term “polar group” refers to a group or substituent that comprises bonds characterized by a dipole moment. Such groups increase solubility of a molecule in a polar solvent such as water. Examples of polar groups include, but are not limited to, hydroxy, thiol, oxo (to form a carbonyl group), carboxy, formyl, amino, amido, urea, carbamate, oxime, imine, sulfoxy, phosphate, glycol and halogen such as fluorine, chlorine, bromine and iodine.

### Compounds of the Invention

In one aspect of the present invention there is provided a compound having formula (I):

Scaffold-[L-(Antigen)ₜ]_{y} (I)

wherein Antigen represents at least a portion of a target antigen for modulating an immune response;
wherein t is 0 or an integer of at least 1;
wherein y is at least 1;
wherein the number of Antigens on the Scaffold is at least 2;
wherein L is a linking group or a covalent bond, wherein when L is a covalent bond, the covalent bond is a single bond attached to a sp or sp² hybridized atom of the Scaffold and when L is a linking group, the linking group is attached to the Scaffold through a single bond attached to a sp or sp² hybridized atom; whereby the Scaffold is sufficiently rigid to maintain the relative position of the single bonds attached to sp or sp² hybridized atoms.

The Scaffold is a rigid or semi-rigid two or three dimensional structure capable of presenting antigens in defined positions or areas of space relative to one another. In some embodiments, the Scaffold comprises a core group or atom that has a defined geometric structure, such as planar, tetrahedral or octahedral, which allows attachment of substituents such as spacers, linkers and antigens with a defined geometry. In some of these embodiments the Scaffold comprises rigid or semi-rigid spacer groups that radiate from the Core and provide a Scaffold with a defined or predictable shape. In some embodiments, a linker bearing an antigen may be attached directly to the Core. In other embodiments, a linker bearing an antigen is attached to a spacer radiating from the Core.

L is a linking group or covalent bond which attaches the antigen to the Scaffold. When L is a covalent bond, L is a single bond to a sp or sp² hybridized atom in the Scaffold. When L is a linking group, L is attached to a sp or sp² hybridized atom of the Scaffold through a single bond. L may be a group that provided the functionality required for attachment of the antigen to the Scaffold. For example, L may be a linking group comprising a carboxylic acid, an amine, an oxime, a heteroaryl group, an amino acid, a dipeptide, a tripeptide or other oligopeptide. The linking group may also reduce congestion at the surface of the Scaffold. This may assist to ensure that the active portion of the antigen is accessible to the immune system to produce an immune response.

A Scaffold may present more than one Antigen where the Antigens presented are the same or a Scaffold may present different Antigens, in some cases many different Antigens.

In another aspect of the invention there is provided a compound having formula (II):

Scaffold[L-(Antigen)ₜ]_{y} (II)

wherein the Scaffold comprises a group

Core-[Spacer]_{z}

wherein the Core is a central atom or group and the spacer is a group wherein each Spacer, alone or in combination with the Core, comprises at least one unbranched or branched moiety comprising at least one group selected from aryl, heteroaryl, alkenyl, acetylenyl and carbonyl,
wherein t is 0 or an integer of at least 1;
wherein y is at least 1;
wherein the number of Antigens on the Scaffold is at least 2;
wherein Antigen represents at least a portion of a target antigen for modulating an immune response;
wherein z is at least 1;
wherein L is a linking group or a covalent bond, wherein when L is a covalent bond, the covalent bond is a single bond attached to a sp or sp² hybridized atom of the spacer and when L is a linking group, the linking group is attached to the spacer(s) through a single bond attached to a sp or sp² hybridized atom; whereby the Scaffold is sufficiently rigid to maintain the relative position of the single bonds attached to sp or sp² hybridized atoms.

In yet another aspect of the invention there is provided a compound having formula (III):

Scaffold-[L-(Antigen)ₜ]_{y} (III)

wherein the Scaffold comprises a group

Core-[Spacer]_{z}

wherein the Core is a central atom or group and the Spacer is a group wherein each Spacer, alone or in combination with the Core, comprises at least one partially conjugated unbranched or branched moiety comprising at least two groups selected from aryl, heteroaryl, alkenyl, acetylenyl and carbonyl,
wherein t is 0 or an integer of at least 1;
wherein y is at least 1;
wherein the number of Antigens on the Scaffold is at least 2;
wherein Antigen represents at least a portion of a target antigen for modulating an immune response;
wherein z is at least 1;
wherein L is a linking group or a covalent bond, wherein when L is a covalent bond, the covalent bond is a single bond attached to a sp or sp² hybridized atom of the spacer and when L is a linking group, the linking group is attached to the spacer(s) through a single bond attached to a sp or sp² hybridized atom; whereby the Scaffold is sufficiently rigid to maintain the relative position of the single bonds attached to sp or sp² hybridized atoms.

In some embodiments of the compounds of the invention the Scaffold comprises a Core and at least one Spacer and the Spacer is bonded to at least one antigen through L. In some embodiments, the compounds include a Core and at least one unbranched Spacer wherein the Spacer, alone or in combination with the Core comprises at least one aryl or heteroaryl group, an alkenyl, acetylenyl or carbonyl group and the Spacer is linked to at least one, especially one or two, antigens through linker(s). In some embodiments, the compounds of the invention include a Core and at least one branched Spacer wherein the Spacer, alone or in combination with the core, comprises at least one aryl or heteroaryl group, an alkenyl, an acetylenyl or carbonyl group and the branched Spacer is bonded to at least one antigen through L.

The Core may be any atom or group that is capable of presenting at least one Spacer in a 2- or 3-dimensional structure. In some embodiments, the Core presents the at least one Spacer, especially more than one Spacer, in a 3-dimensional structure.

In some embodiments the Core is selected from a metal ion, an aryl group, a heteroaryl group, a conjugated macrocyclic group such as a porphyrin, or an organometallic complex, a tetrahedral carbon atom, a tetrahedral silicon atom, a silsesquioxane or a caged hydrocarbon group such as adamantyl. In some embodiments, the Core may include acetylenyl or alkenyl, especially vinyl, substituents to which Spacer moieties are attached.

In particular embodiments, the Core is an aryl or heteroaryl group, an acetylenyl or vinyl group, a caged hydrocarbon group or a caged silicon group containing group such as a silsesquioxane. Suitable acetylenyl Cores may be substituted with one or two Spacers. Suitable vinyl Cores may be substituted with one to four, especially two to four Spacers, more especially two or four spacers. When substituted with two to four Spacers, the Core vinyl group may be a mixture of geometric isomers or a single geometric isomers and the Spacers may be E or Z in relation to one another. Suitable aryl and heteroaryl Cores include one or more Spacers with a maximum number equal to the number of atoms in the ring capable of substitution. For example, a heteroaryl pyridine ring has six substitutable atoms capable of bearing a Spacer, a phenyl ring has six substitutable atoms capable of bearing a Spacer, a furan ring has four substitutable atoms capable of bearing a Spacer and a pyrrole ring has five substitutable atoms capable of bearing a Spacer. In particular embodiments, the Core is a phenyl ring bearing one to six Spacer moieties, especially two to six, three to six or four to six Spacer moieties, more especially three or six Spacer moieties. Suitable caged hydrocarbons include, but are not limited to, cubane, prismane and adamantane and these caged hydrocarbons include at least one Spacer and up to a maximum of Spacers equal to the number of substitutable carbon atoms. In one embodiment, the caged hydrocarbon is an adamantyl group substituted with one to four, especially four Spacer groups, particularly substituted at the ring fusing carbon atoms. Suitable caged silicon containing Cores include silsesquioxane which may be substituted with 1 to 8, 2 to 8, 3 to 8, 4 to 8, 5 to 8, 6 to 8, 7 to 8, or 8, especially 8 Spacers at one to all of the silicon atoms.

In some embodiments the Core is bonded to the Spacer through a bond to a sp or sp² hybridized carbon atom of the Spacer. This may be the carbon atom of an alkenyl, alkynyl or carbonyl group or an aryl or heteroaryl group, which is the beginning of the Spacer.

In some embodiments, the Core is a phenyl ring, an acetylenyl group, an adamantyl group or a silsesquioxane group.

In some embodiments, the sp or sp² hybridized atom that is at the bond that links the Core to the Spacer is part of an acetylenyl, alkenyl or carbonyl group that is conjugated with the Core. In some embodiments, the Core is an aryl or heteroaryl group and the sp or sp² hybridized atom is part of an acetylenyl group, vinyl group or carbonyl group of the Spacer. In some embodiments, the Core is a phenyl group substituted with three or six Spacers that are attached to the Core through an acetylenyl, vinyl or carbonyl group. When the phenyl group is substituted with three Spacers, they are located in the 1,2,3 positions, 1,2,4 positions, 1,2,5 positions, 1,3,4 positions or 1,3,5 positions, especially in the 1,3,5 positions.

In some embodiments, the sp or sp² hybridized atom that is at the bond that links the Core to the Spacer is part of an aryl or heteroaryl group, especially an aryl group, more especially a phenyl group.

In some embodiments, the Spacer is an unbranched, at least partially conjugated moiety that is capable of being substituted with at least one antigen. This Spacer, alone or together with the Core is an unbranched at least partially conjugated moiety comprises at least two aryl, heteroaryl, acetylenyl, alkenyl or carbonyl groups. When the unbranched Spacer contains only an acetylenyl, alkenyl or carbonyl group, the group is at least partially conjugated with the Core and the Core is an aryl, heteroaryl, vinyl or acetylenyl group.

An unbranched Spacer may be linear or non-linear. For example if an unbranched Spacer comprises two phenyl groups linked directly to one another, the second phenyl group may be linked to the first phenyl group in the 4 position with respect to the attachment of the first phenyl group to the Core, to provide a linear unbranched Spacer. Alternatively, the second phenyl group may be linked to the first phenyl group in a position other than the 4 position with respect to the attachment to the first phenyl group to the Core, to provide a non-linear unbranched Spacer.

In some embodiments, at least some of the unbranched Spacers bear one or more antigens. In some embodiments, each unbranched Spacer bears one or more antigens which may be the same or different. In particular embodiments, each unbranched Spacer bears 1 or 2 antigens. In particular embodiments, the antigen or antigens are located on the distal end of the Spacer with respect to the Core. In some embodiments, some Spacers have no antigen bound to them or are bound to a moiety other than an antigen.

In some embodiments, the unbranched Spacer is substituted with further substituents that increase solubility in a solvent or carrier such as water. Suitable substituents include, but are not limited to polar substituents such as hydroxyl, amino, thiol, carboxy, glycol or sulfoxy. In some embodiments the unbranched Spacer is substituted with further substituents that improve immunogenicity such as T-helper peptide sequences, PEG or lipid groups such as the PAM₃-Cys unit, that are designed to mimic bacterial lipid groups that act as adjuvants to the immune system.

In some embodiments, the Spacer is a branched Spacer, for example, a dendron. The branched Spacer includes one or more aryl, heteroaryl or alkenyl groups bearing two substituents in addition to the bond to the Core. In some embodiments, the branched Spacer may have 1 to 5 generations of branching, especially 1 to 4 generations, 1 to 3 generations, 1 or 3 generations, more especially 1 or 2 generations of branching.

At least one branch of the Spacer bears an -L-Antigen group. In some embodiments, a branched Spacer may bear 1 to 36-L-Antigen groups, 1 to 16-L-Antigen groups, 1 to 8-L-Antigen groups, 1 to 4-L-Antigen groups or 1 to 2-L-Antigen groups, especially 1 to 4 or 1 to 2-L-Antigen groups. In some embodiments, some branches of the Spacer moiety bear no Antigen or bear a moiety other than an antigen.

When a branched Spacer bears more than one -L-antigen group, the -L- may be the same or different and/or the antigen may be the same or different.

In some embodiments, at least one antigen is located at the distal end of a branch of the Spacer with respect to the Core.

In some embodiments the compound is a compound of formula (V):

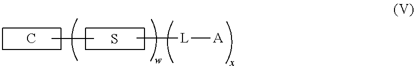

wherein C is an aryl or heteroaryl group, a caged hydrocarbon group, a caged silicon containing group, an acetylenyl group or a vinyl,
S is selected from aryl, heteroaryl, acetylenyl, alkenyl or carbonyl or a group:

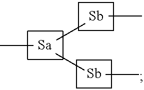

Sa is selected from aryl, heteroaryl, acetylenyl or carbonyl;
Sb is selected from aryl, heteroaryl, acetylenyl or carbonyl or is a group:

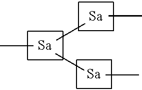

L is a covalent bond or a linking group;
A represents at least a portion of a target antigen for modulating an immune response;
w is an integer from 1 to 6; and
x is at least 2.

In some embodiments, the Core is selected from phenyl, hexaphenylbenzene, 1,3,5-triphenylbenzene, adamantane, 1,1′,1″,1′″-tetraphenyladamantane, silsesquioxane, octavinylsilesquioxane, acetylene and 1,3,5-tricarbonylbenzene.

In some embodiments, the Spacer is selected from phenyl, 4-biphenyl, 3,5-diphenylbenzene, 3,5-dithiophen-ylbenzene, 4-[3,5-diphenylbenzene]benzene, ethynyl, 2-phenylethynyl and ethenyl.

In some embodiments, L is a covalent bond or a linking group is selected from:

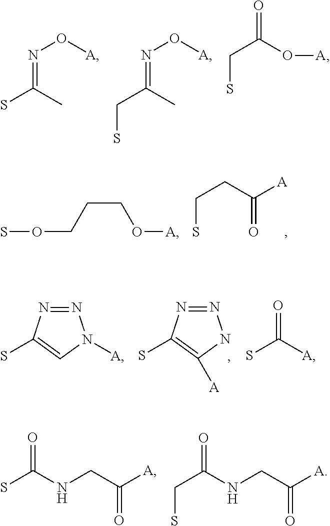

In some embodiments where the Core is a phenyl group with three substituents, they are in the 1, 3 and 5 positions.

In some embodiments where the Spacer comprises a phenyl ring substituted with two substituents and the substituents are in the 3 and 5 positions with respect to the bond to the Core or previous group in the spacer.

In a particular embodiment in which the Spacer is branched, the compound of the invention is a compound having the formula (IV):

CORE-[DENDRITE]n (IV)

wherein CORE represents an atom or group, n represents an integer of at least 1, and DENDRITE, which may be the same or different if n is greater than 1, represents an at least partly conjugated dendritic molecular structure comprising groups selected from aryl, heteroaryl, alkenyl, acetylenyl and carbonyl, and said DENDRITE comprising at least one antigen; and wherein said CORE terminating at a bond to a sp² hybridized atom which forms part of a moiety that has at least two substituents.

In some embodiments of formula (IV), the CORE is an aryl group, heteroaryl, caged hydrocarbon group or caged silicon containing group optionally substituted with one or more vinyl or acetylenyl groups, especially an aryl group optionally substituted with one or more vinyl or acetylenyl groups, more especially a phenyl group optionally substituted with one to six vinyl or acetylenyl groups, most especially a phenyl group.

The DENDRITE may be any branched dendritic structure comprising groups selected from aryl, heteroaryl, alkenyl and acetylenyl groups or mixtures thereof, which are at least partially conjugated. In some embodiments, the DENDRITE comprises a dendritic structure that is fully conjugated. In some embodiments, the DENDRITE comprises diaryl substituted aryl groups, diaryl substituted heteroaryl groups, diheteroaryl substituted aryl groups, diheteroaryl substituted aryl groups, aryl-heteroaryl substituted aryl groups or aryl-heteroaryl substituted heteroaryl groups where each aryl or heteroaryl substituent is linked to the aryl or heteroaryl group directly or through an intervening vinyl or acetylenyl group. In each of these cases, the disubstitution of the aryl or heteroaryl groups is in addition to the bond to the Core, a bond to a lower generation of branching, a previous group in the Spacer or the bond to the Antigen. In particular embodiments, the aryl or heteroaryl substituents are linked directly to the aryl or heteroaryl group.

In some embodiments, the DENDRITE has 1 to 5 generations of branching, especially 1 to 4 generations, 1 to 3 generations or 1 to 2 generations of branching, more especially 1 to 2 generations of branching.

The DENDRITE comprises at least one antigen, especially 1 to 36 antigens, 1 to 18 antigens, 1 to 8 antigens, 1 to 4 antigens or 1 to 2 antigens, more especially 1 to 4 antigens or 1 to 2 antigens.

When the DENDRITE comprises more than one antigen, the antigens may be the same or different.

In some embodiments, the antigen forms the first generation or second generation branching. In particular embodiments, at least one antigen is located at the distal end of the DENDRITE with respect to the CORE.

In some embodiments, the aryl, heteroaryl and/or alkenyl groups of the DENDRITE are substituted with further substituents that increase solubility in a solvent or carrier such as water. Suitable substituents include polar substituents such as hydroxy, amino, thiol, carboxy, glycol and sulfoxy. In some embodiments, the aryl, heteroaryl and/or alkenyl groups of the DENDRITE are substituted with further substituents that improve immunogenicity such as T-helper peptide sequences, PEG or lipid groups such as the PAM₃-Cys unit, that are designed to mimic bacterial lipid groups that act as adjuvants to the immune system.

The term “n” denotes the number of DENDRITEs attached to the CORE. The maximum number of DENDRITEs attached to the CORE is determined by the structure of the CORE. For example, when the CORE comprises a phenyl group, the maximum number of DENDRITEs attached to the CORE is six. When the CORE comprises a tetrahedral carbon or silicon atom, the maximum number of DENDRITEs is 4. When the CORE comprises a silesquioxane group, the maximum number of DENDRITEs is 8. When the CORE comprises a naphthyl group or a phenanthrene group, the maximum number of DENDRITEs is 8 and 10 respectively. When the CORE is a metal ion or an organometallic complex, the maximum number of DENDRITEs is determined by the valency of the metal ion or atoms of the ligands in an organometallic complex capable of substitution. In some embodiments, n is an integer from 1 to 12, especially 1 to 10, 1 to 8 or 1 to 6.

In some embodiments where n is greater than 1, the DENDRITEs may be the same or different and may differ in the branching of the DENDRITE and/or the linking of the components of the DENDRITE and/or the antigens incorporated into the DENDRITE.

Suitable rigid at least partially conjugated scaffolds that have functionalization or are capable of being functionalized so that they may be coupled to an antigen and are therefore suitable for use in the present invention include dendrimers described in Lo and Burn, Chem. Rev., 2007:107:1097-1116 and Jiang et al., Organic Letters. 2007, 9(22):4539-4542.

In some embodiments, the compound of formula (IV) is a compound of formula (VI):

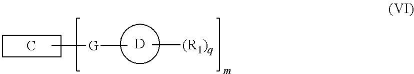

wherein C is an aryl or heteroaryl group;
G is absent or is selected from the group consisting of —C(O)—, —CR₂═CR₃— or —C≡C—;
each R₁ is independently selected from

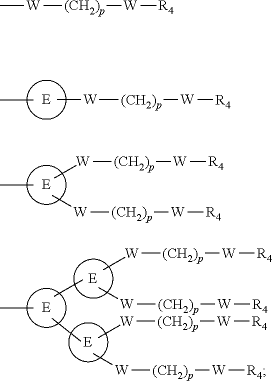

R₂ is hydrogen, alkyl or a polar group,
R₃ is hydrogen, alkyl, a polar group or

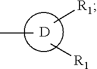

D is an aryl or heteroaryl group;
each E is independently selected from an aryl or heteroaryl group;
each R₄ is the same or different and is an antigen and its attachment to D or E;
each W is independently absent or is independently selected from —O—, —NH—, —S—, —C(O)—, —S(O)— or S(O)₂;
each p is the same or different and is 0 or an integer from 1 to 10;
q is an integer of at least 2; and
m is an integer from 1 to 10.

In some embodiments of the compound of formula (VI), one or more of the following applies:

C is an aryl group, especially a phenyl group or naphthyl group, more especially a phenyl group.
G is absent or is —CH═CH— or —C≡C— especially where G is absent or is —CH═CH—, more especially where L is absent;
D is an aryl group, especially a phenyl or naphthyl group, more especially a phenyl group;
R₁ is selected from

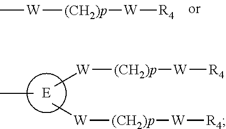

each E is independently selected from an aryl group selected from phenyl or naphthyl or a heteroaryl group selected from furanyl, thienyl, benzothienyl, benzofuranyl, pyrazinyl, pyridazinyl, pyridinyl, pyrimidinyl, pyrrolyl and thiazolyl, especially phenyl, thienyl, furanyl, pyrrolyl and pyridinyl;
each W is independently absent or selected from —O—, —C(O)O—, —S—, or —NH—, especially where W is independently selected from being absent, —C(O)O— or —O—;
each p is 0 or an integer from 1 to 6, especially 0 or an integer from 1 to 3;
q is at least 2 and up to the maximum number of attachment points on D, especially 2 to 5, more especially 2 to 3, most especially 2;
each m is an integer from 1 to 8, especially 1 to 6;
when D is phenyl, each R₁ substituent is in the meta (3 and 5) position of D with respect to the attachment to L or C; and
when E is phenyl, the two substituents are in the meta position (3 and 5) with respect to the attachment to D.

Exemplary compounds of the invention include:

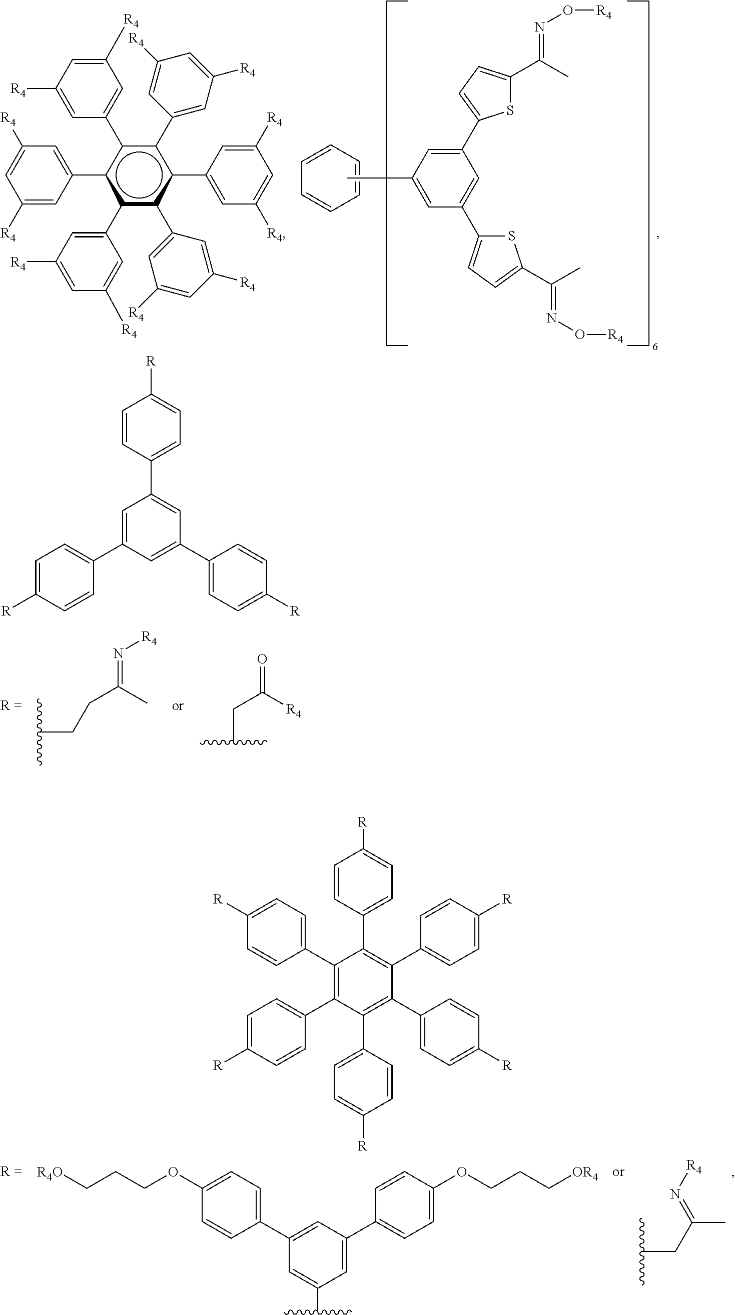

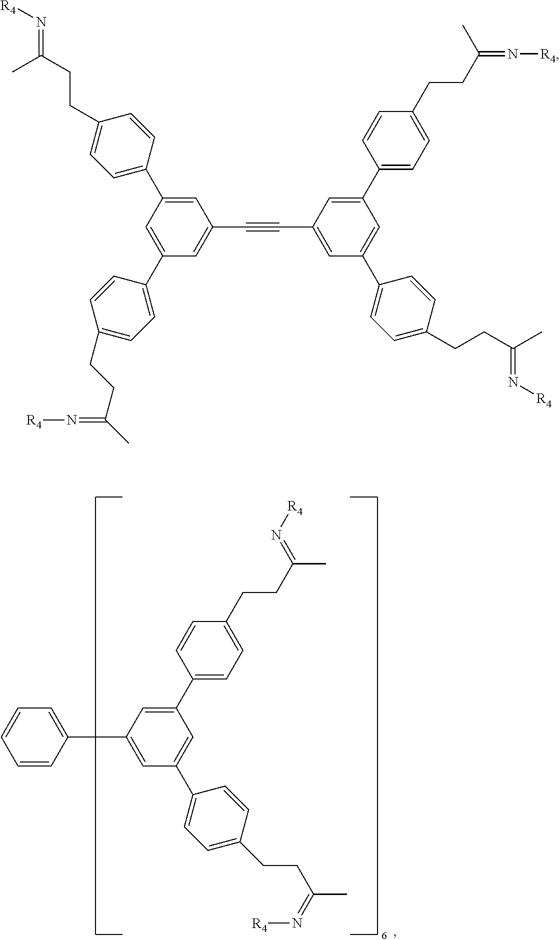

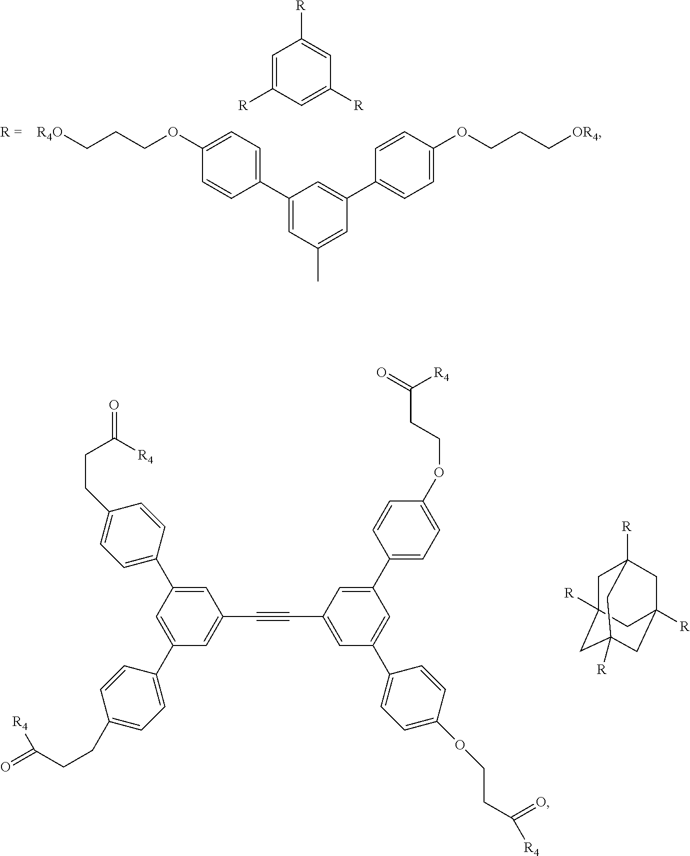

wherein each R is

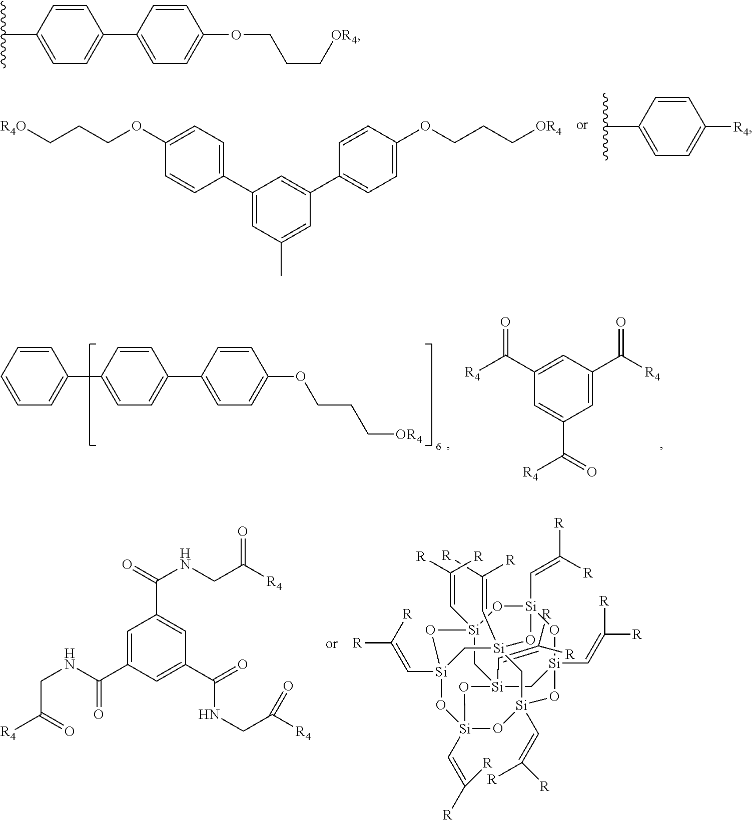

wherein each R is H or is one of

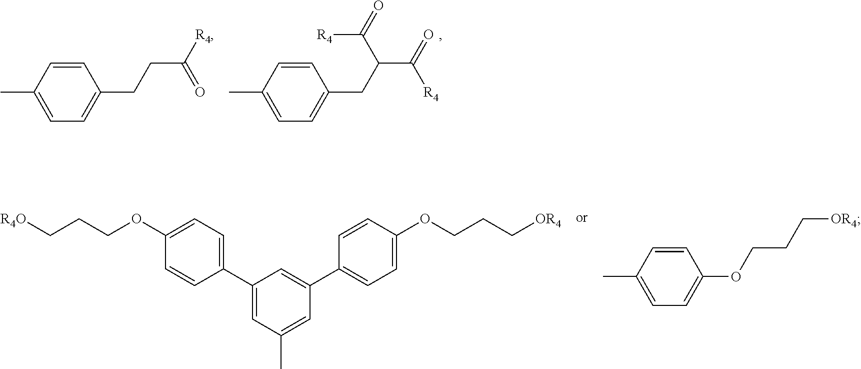

wherein in each compound when R is not H, the R groups are the same, and wherein at least some R groups are not H; and
where each R₄ in the above structural formulae is the same or different and is an antigen and/or an attachment to an antigen, such as a covalent bond, an ester, an amide, ether, oxime or heteroaryl group.
Antigens and their Use

Suitably, each antigen corresponds to at least a portion of a corresponding target antigen to which modulation (e.g., stimulation, enhancement or attenuation) of an immune response is desired. For example, the target antigen may be selected from foreign or endogenous antigens as described for example below, and can be any type of biological molecule including, for example, simple intermediary metabolites, sugars, lipids, and hormones as well as macromolecules such as complex carbohydrates, phospholipids, nucleic acids, polypeptides and peptides.

Target antigens may be selected from endogenous antigens produced by a host or exogenous antigens that are foreign to the host. Suitable endogenous antigens include, but are not restricted to, self-antigens that are targets of autoimmune responses as well as cancer or tumour antigens. Illustrative examples of self antigens useful in the treatment or prevention of autoimmune disorders include, but not limited to, diabetes mellitus, arthritis (including rheumatoid arthritis, juvenile rheumatoid arthritis, osteoarthritis, psoriatic arthritis), multiple sclerosis, myasthenia gravis, systemic lupus erythematosus, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing haemorrhagic encephalopathy, idiopathic bilateral progressive sensorineural hearing loss, aplastic anaemia, pure red cell anaemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, and interstitial lung fibrosis. Other autoantigens include those derived from nucleosomes for the treatment of systemic lupus erythematosus (e.g., GenBank Accession No. D28394; Bruggen et al., 1996, Ann. Med. Interne (Paris), 147:485-489) and from the 44,000 Da peptide component of ocular tissue cross-reactive with O. volvulus antigen (McKeclmie et al., 1993, Ann Trop. Med. Parasitol. 87:649-652). Thus, illustrative autoantigens antigens that can be used in the compositions and methods of the present invention include, but are not limited to, at least a portion of a lupus autoantigen, Smith, Ro, La, U1-RNP, fibrillin (scleroderma), pancreatic β cell antigens, GAD65 (diabetes related), insulin, myelin basic protein, myelin proteolipid protein, histones, PLP, collagen, glucose-6-phosphate isomerase, citrullinated proteins and peptides, thyroid antigens, thyroglobulin, thyroid-stimulating hormone (TSH) receptor, various tRNA synthetases, components of the acetyl choline receptor (AchR), MOG, proteinase-3, myeloperoxidase, epidermal cadherin, acetyl choline receptor, platelet antigens, nucleic acids, nucleic acid:protein complexes, joint antigens, antigens of the nervous system, salivary gland proteins, skin antigens, kidney antigens, heart antigens, lung antigens, eye antigens, erythrocyte antigens, liver antigens and stomach antigens.

Non-limiting examples of cancer or tumour antigens include antigens from a cancer or tumour selected from ABL1 protooncogene, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, dermatofibrosarcoma-protuberans, desmoplastic-small-round-cell-tumour, ductal carcinoma, endocrine cancers, endometrial cancer, ependymoma, oesophageal cancer, Ewing's Sarcoma, Extra-Hepatic Bile Duct Cancer, Eye Cancer, Eye: Melanoma, Retinoblastoma, Fallopian Tube cancer, Fanconi anaemia, fibrosarcoma, gall bladder cancer, gastric cancer, gastrointestinal cancers, gastrointestinal-carcinoid-tumour, genitourinary cancers, germ cell tumours, gestational-trophoblastic-disease, glioma, gynaecological cancers, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukaemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumours, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-Macroglobulinemia, Wilms' Tumour. In certain embodiments, the cancer or tumour relates to melanoma. Illustrative examples of melanoma-related antigens include melanocyte differentiation antigen (e.g., gp100, MART, Melan-A/MART-1, TRP-1, Tyros, TRP2, MC1R, MUC1F, MUC1R or a combination thereof) and melanoma-specific antigens (e.g., BAGE, GAGE-1, gp100In4, MAGE-1 (e.g., GenBank Accession No. X54156 and AA494311), MAGE-3, MAGE4, PRAME, TRP21N², NYNSO1a, NYNSO1b, LAGE1, p97 melanoma antigen (e.g., GenBank Accession No. M12154) p5 protein, gp75, oncofetal antigen, GM2 and GD2 gangliosides, cdc27, p21ras, gp100^{Pmel117} or a combination thereof. Other tumour-specific antigens include, but are not limited to: etv6, amll, cyclophilin b (acute lymphoblastic leukemia); Ig-idiotype (B cell lymphoma); E-cadherin, α-catenin, β-catenin, γ-catenin, p120ctn (glioma); p21 ras (bladder cancer); p21 ras (biliary cancer); MUC family, HER2/neu, c-erbB-2 (breast cancer); p53, p21ras (cervical carcinoma); p21ras, HER2/neu, c-erbB-2, MUC family, Cripto-lprotein, Pim-1 protein (colon carcinoma); Colorectal associated antigen (CRC)—CO17-1A/GA733, APC (colorectal cancer); carcinoembryonic antigen (CEA) (colorectal cancer; choriocarcinoma); cyclophilin b (epithelial cell cancer); HER2/neu, c-erbB-2, ga733 glycoprotein (gastric cancer); α-fetoprotein (hepatocellular cancer); Imp-1, EBNA-1 (Hodgkin's lymphoma); CEA, MAGE-3, NY-ESO-1 (lung cancer); cyclophilin b (lymphoid cell-derived leukemia); MUC family, p21ras (myeloma); HER2/neu, c-erbB-2 (non-small cell lung carcinoma); Imp-1, EBNA-1 (nasopharyngeal cancer); MUC family, HER2/neu, c-erbB-2, MAGE-A4, NY-ESO-1 (ovarian cancer); Prostate Specific Antigen (PSA) and its antigenic epitopes PSA-1, PSA-2, and PSA-3, PSMA, HER2/neu, c-erbB-2, ga733 glycoprotein (prostate cancer); HER2/neu, c-erbB-2 (renal cancer); viral products such as human papilloma virus proteins (squamous cell cancers of the cervix and oesophagus); NY-ESO-1 (testicular cancer); and HTLV-1 epitopes (T cell leukemia). In other embodiments, the cancer or tumour antigens are carbohydrate antigens, illustrative examples of which include: glycolipid structures such as globo-H (Fucα2Galβ3GalNAcβ3Galα4LacβCer), gangliosides: GM1 Galβ3GalNAcβ4(NeuNAcα3)LacβCer or GD2 GalNAcβ4(NeuNAcα8NeuNAcα3)LacβCer; Lewis-type fucosylated structures such as Lewis a and x: Galβ3/4(Fucα4/3)GlcNAc, Lewis y: Fucα2Galβ4(Fucα3)GlcNAc, sialyl-Lewis x: NeuNAcα3Galβ4(Fucα3)GlcNAc, and some combinations of these on polylactosamine chains; O-glycan core structures, such as T-antigen Galβ3GalNAcαSer/Thr-Protein, Tn-antigen GalNAcαSer/Thr-Protein or sialyl Tn-antigen NeuNAcα6GalNAcαSer/Thr-Protein. In specific embodiments, the cancer or tumour associated oligosaccharide is selected from: GlcNAcβ2Man, GlcNAcβ2Manα3 (GlcNAcβ2Manα6)Man, GlcNAcβ2Manα3 (GlcNAcβ2Manα6)Manβ4 GlcNAc, GlcNAcβ2Manα3 (GlcNAcβ2Manα6)Manβ4GlcNAcβ4-GlcNAc, GlcNAcβ2Manα3 (GlcNAcβ2 Manα6)Manβ4 GlcNAc-β4 (Fucα6)GlcNAc, GlcNAcβ2Manα3 (Manα6)Man, GlcNAcβ2Manα3 (Manα6)Manβ4 GlcNAc, GlcNAcβ2Manα3 (Manα6)Manβ4GlcNAcβ4GlCNAc, GlcNAcβ2Manα3 (Manα6)Manβ4GlcNAcβ4(Fucα6)-GlcNAc, Manα3(GlcNAcβ2Manα6)Man, Manα3(GlcNAcβ32Manα6)Manβ4GlcNAc, Manα3 (GlcNAcβ2 Manα6)Manβ4GlcNAcβ4GlcNAc, Manα3(GlcNAcβ2Manα6)Manβ4GlcNAcβ4(Fucα6)-GlcNAc, GlcNAcβ2Manα3 Man, GlcNAcβ2Manα3Manβ4GlcNAc, GlcNAcβ2Manα3Manβ4GlcNAcβ4GlcNAc, GlcNAcβ2Manα3 Manβ4 GlcNAcβ4 (Fucα6)GlcNAc, GlcNAcβ2Manα6Man, GlcNAcβ2Manα6Manα4GlcNAc, GlcNAcβ2Manα6Manβ4GlcNAcβ4GlcNAc, or GlcNAcβ2Manα6Manβ4GlcNAcβ4(Fucα6)GlcNAc

Foreign antigens are suitably selected from transplantation antigens, allergens as well as antigens from pathogenic organisms. Transplantation antigens can be derived from donor cells or tissues from e.g., heart, lung, liver, pancreas, kidney, neural graft components, or from the donor antigen-presenting cells bearing MHC loaded with self antigen in the absence of exogenous antigen.

Non-limiting examples of allergens include Fel d 1 (i.e., the feline skin and salivary gland allergen of the domestic cat Felis domesticus, the amino acid sequence of which is disclosed International Publication WO 91/06571), Der p I, Der p II, Der fI or Der fII (i.e., the major protein allergens from the house dust mite dermatophagoides, the amino acid sequence of which is disclosed in International Publication WO 94/24281). Other allergens may be derived, for example from the following: grass, tree and weed (including ragweed) pollens; fungi and moulds; foods such as fish, shellfish, crab, lobster, peanuts, nuts, wheat gluten, eggs and milk; stinging insects such as bee, wasp, and hornet and the chirnomidae (non-biting midges); other insects such as the housefly, fruitfly, sheep blow fly, screw worm fly, grain weevil, silkworm, honeybee, non-biting midge larvae, bee moth larvae, mealworm, cockroach and larvae of Tenibrio molitor beetle; spiders and mites, including the house dust mite; allergens found in the dander, urine, saliva, blood or other bodily fluid of mammals such as cat, dog, cow, pig, sheep, horse, rabbit, rat, guinea pig, mouse and gerbil; airborne particulates in general; latex; and protein detergent additives.

Exemplary pathogenic organisms include, but are not limited to, viruses, bacteria, fungi parasites, algae and protozoa and amoebae. Illustrative examples of viruses include viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus, herpesviruses, papillomavirus, Ebola virus, influenza virus, Japanese encephalitis (e.g., GenBank Accession No. E07883), dengue (e.g., GenBank Accession No, M24444), hantavirus, Sendai virus, respiratory syncytial virus, othromyxoviruses, vesicular stomatitis virus, visna virus, cytomegalovirus and human immunodeficiency virus (HIV) (e.g., GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C, viral components such as hepatitis C viral RNA. Illustrative examples of influenza viral antigens include; but are not limited to, antigens such as hemagglutinin and neurarninidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens. In specific embodiments, the viral antigen is a carbohydrate antigen, non-limiting examples of which include oligo-D-mannose moiety of HIV and poly-β-1,6GlcNAc of Staphylococcus aureus polysaccharide intercellular adhesion (PIA).

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, illustrative fungal antigens that can be used in the compositions and methods of the present invention include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Non-limiting examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae.), cholera (e.g., Vibrio cholerae), anthrax (e.g., Bacillus anthracia), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487), Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pnermiococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. LOS198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-5-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 561cDa antigen and other trypanosomal antigen components.

The present invention also contemplates toxin components as antigens. Illustrative examples of toxins include, but are not restricted to, staphylococcal enterotoxins, toxic shock syndrome toxin; retroviral antigens (e.g., antigens derived from HIV), streptococcal antigens, staphylococcal enterotoxin-A (SEA), staphylococcal enterotoxin-B (SEB), staphylococcal enterotoxin₁₋₃ (SE₁₋₃), staphylococcal enterotoxin-D (SED), staphylococcal enterotoxin-E (SEE) as well as toxins derived from mycoplasma, mycobacterium, and herpes viruses.

The antigen(s) may be selected from proteinaceous antigens, lipid antigens, glycolipid antigens and carbohydrate antigens. In some embodiments, the antigen is a proteinaceous antigen, illustrative examples of which include peptide and polypeptide antigens. Non-limiting antigenic peptides are typically at least 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues in length and suitably no more than about 500, 200, 100, 80, 60, 50, 40 amino acid residues in length. The length of the peptides may be selected to enhance the production of a cytolytic T lymphocyte response (e.g., peptides of about 6 to about 10 amino acids in length), or a T helper lymphocyte response (e.g., peptides of about 12 to about 20 amino acids in length). In some embodiments, a peptide sequence is derived from at least about 5, 10, 15, 20, 30, 40, 50, 60, 70, 80, 90, 99% (and all integer percentages therebetween) of the sequence corresponding to a corresponding target antigen.

In other embodiments, the antigen is a carbohydrate antigen. The carbohydrate antigen may be selected from saccharide residues, illustrative examples of which include monosaccharides, disaccharides, and oligosaccharides such as but not limited to trisaccharides and saccharides including 2-10 monosaccharide units. In some embodiments, the saccharide residue includes at least one hexose residue, such as allose, altrose, glucose, mannose, gulose, idose, galactose, talose and combinations thereof. In some embodiments, a saccharide residue is a pentose, tetrose or triose. A saccharide residue may contain a combination of hexose, pentose, tetrose and triose residues. Further, in some embodiments, a saccharide residue may contain a 7-, 8-, 9,10-, 11-, 12- or more carbon saccharide, such as sialic acid.

Exemplary saccharide residues include pyranose forms. A saccharide residue includes D- and L-aldopyranoses, D- and L-aldofuranoses, D- and L-ketopyranoses and D- and L-ketofuranoses. In some embodiments, a saccharide residue further includes modified saccharide residues such as deoxy derivatives, including dexoyamine, deoxythio-, and deoxyhalo-saccharides. A saccharide residue further includes acid derivatives of saccharide residues described above, such as glucuronic acid and galacturonic acid. In addition, a saccharide residue includes glycosyl residues such as N-acetylneuraminic acid (sialic acid).

Saccharides may include substituents replacing an alcoholic hydroxy group or the hydrogen atom of an alcoholic hydroxy group of a saccharide or saccharide derivative. Such substituents include COOH, sialic acid, NHAc, C₁₋₈ acyl, anhydro, C₁₋₈ alkyl, NH₂, halogen, OSO₃D, OPO₃D, CH₂OH, CH₂OSO₃D, or CH₂OPO₃D. Further, a substituent may be a saccharyl group.

The saccharides may include an O-substituent, that is, in which a substituent replaces the hydrogen atom of an alcoholic hydroxy group of a saccharide or saccharide derivative. Examples of such substituents include alkyl-, acyl-, and phosphorus containing-groups. In specific embodiments, the substituents include the sulfur moieties (DO)S(O)₂— or (O⁻)S(O)₂—, bonded to oxygen, where D is H or a cation.

Where multiple monosaccharide residues are included in a saccharide, they are suitably linked by α-O-glycosidic or β-O-glycosidic linkage. Further, S-, N- and C-glycosidic linkages are optionally included. An illustrative optional glycosidic link is an S-linkage since this is more resistant to glycosidases than a corresponding O-glycosidic bond between monosaccharide units. Typical bonds include (1→2), (1→3)-, (1→4)-, (1→5)-, (1→6)-, (2→3)- and (2→6) glycosidic linkages. One of skill in the art will recognize that where a monosaccharide having more than 6 carbons is present, further typical bonds are present, such as (2→8) for instance.

In some embodiments of the invention, the antigen is an antigen capable of eliciting an immunogenic response to HIV. One example of such an antigen is the discontinous antigen from the HIV1 envelope protein, gp120. This antigen is designated as CG10 and is composed of discontinuous peptide segments brought together by folding of the protein. These component sequences (SEQ ID NO: 1-3) can be displayed on a construct and together constitute the complete antigen.

| | |
|---|---|
| Cys Val Lys Leu Thr | SEQ ID NO: 1 |
| Val Gly Lys Ala Met Tyr | SEQ ID NO: 2 |
| Cys Pro Lys Glu Phe Lys Gln Ile | SEQ ID NO: 3 |

In some embodiments, the antigen is an antigen capable of eliciting an immunogenic response to Staphylococcus spp, especially Staphyloccus aureus. One surface carbohydrate of S. aureus is the polysaccharide intercellular adhesion (PIA). PIA is a linear polymer of β-1,6-linked glucoaminoglycan with at least 130 residues and a molecular weight of ˜28 kDa. The disaccharide repeat unit can be synthesized and displayed multiple times on a construct to mimic the structure of PIA and elicit antibodies that will recognize native PIA. This disaccharide antigen is:

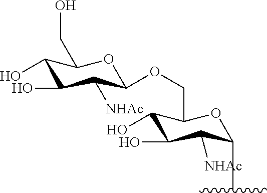

### Methods of Preparing the Compounds of the Invention

With dendrimer chemistry, it is possible to construct macromolecules with tight control of size, shape topology, flexibility and reactive end groups. In what is known as divergent synthesis, these macromolecules start by reacting an initiator core in high-yield iterative reaction sequences to build symmetrical branches radiating from the core with well-defined reactive end groups. Alternatively, in what is known as convergent synthesis, dendritic wedges are constructed separately then several dendritic wedges are coupled at the focal points with a polyfunctional core.

Divergent dendritic syntheses form concentric layers, known as generations, with each generation increasing the molecular mass and the number of reactive groups at the branch ends. The problem with this strategy is that at higher generations not all of the reactive groups react leading to defects in a branch or branches of the dendrimer. The convergent route overcomes this problem as at each generation there is always a small number of reactions that need to be carried out and if a branch is not added then the structure is easier to purify. A convergent route therefore leads to a simpler method of forming the dendrimer in a highly pure, uniform monodisperse macromolecule that solubilizes readily over a range of conditions. As dendrimers grow with each generation, the steric constraints from congestion of the branches force the shape of the macromolecule to change from a starfish-shaped molecule to a globular molecule. For example, with StarBurst™ polyamidoamine (“PAMAM”) dendrimers, generations 0-3 are dome-shaped, generation 4 is a transition generation with an oblate spheroid shape, and generations 5 and greater are symmetrically spherical with a hollow interior and a surface skin. This change of shape, from domes to spheres, with increasing size (caused by increasing surface congestion at the branch ends) is a general feature of dendritic macromolecule made up of flexible spacers. The problem with such dendrimers for presenting antigens in specific directions in space is that only at high generations is any structural order achievable and high generation materials may not be suitable for manufacturing.

The peripheral functional groups of dendrimers can be used to link multiple labels (e.g. biotin, fluorophores, or combinations thereof) to other molecules such as DNA oligomers. Alternatively, multiple macromolecules such as peptides, nucleic acids and carbohydrates, or combinations thereof, can be linked to the dendrimers. The ability to link the same or different molecules of choice at the periphery of the dendrimer provides for signal amplification potential.

The compounds of the invention may be conveniently prepared by convergent synthesis. This has the advantages of ensuring that a single compound is prepared, the number of antigens presented on the Spacer or DENDRITEs is known and that, if required, different antigens can be introduced into a single compound. The compounds of the invention of low generations (one or two) or those that are unbranched can also be conveniently prepared by a divergent route or a combination of convergent and divergent steps.

In a first step, a diaryl-aryl bromide or iodide, diaryl-heteroaryl bromide or iodide, (aryl)(heteroaryl)-aryl bromide or iodide, (aryl)(heteroaryl)-heteroaryl bromide or iodide or (heteroaryl)(heteroaryl)-aryl bromide or iodide is prepared as shown in scheme 1:

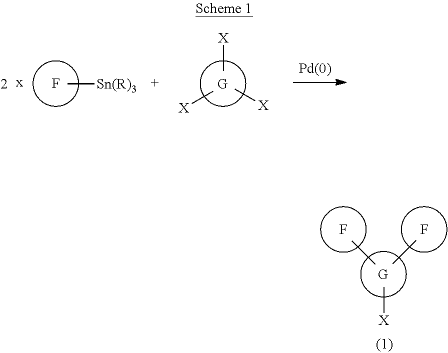

In scheme 1, each F is an aryl or heteroaryl group, G is an aryl or heteroaryl group, X is a bromide or iodide and each R is an alkyl group such as n-butyl. The reaction occurs using Stille coupling conditions with a Pd(0) catalyst.

A monoaryl-aryl bromide or iodide, heteroaryl-aryl bromide or iodide, aryl-heteroaryl bromide or iodide or heteroaryl-aryl bromide or iodide can be prepared in a similar manner using only one equivalent of trialkyl tin compound to provide an unbranched compound. Compound (1), whether mono or disubstituted, may be treated to introduce further functionalization on the G ring as shown in Scheme 2 (shown as disubstituted):

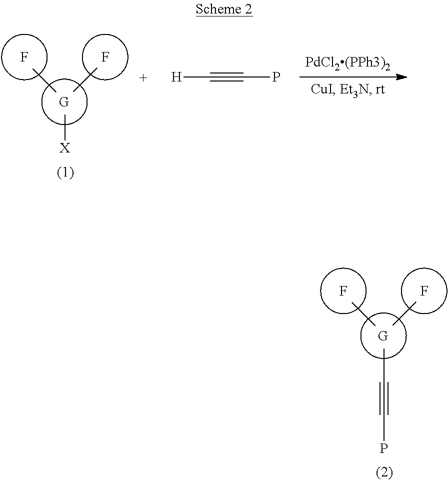

In scheme 3, the group P is a protecting group and the acetylene is coupled with a mono or disubstituted aryl or heteroaryl group G (shown as disubstituted), using a Sonogashira reaction. After deprotection of the acetylene group, compound (2) can undergo a second Sonogashira reaction to provide coupling to a second compound (1). The G and F groups in the second compound (1) may be the same or different from the first compound (1) to provide a compound (3) as shown in Scheme 3 (shown as disubstituted):

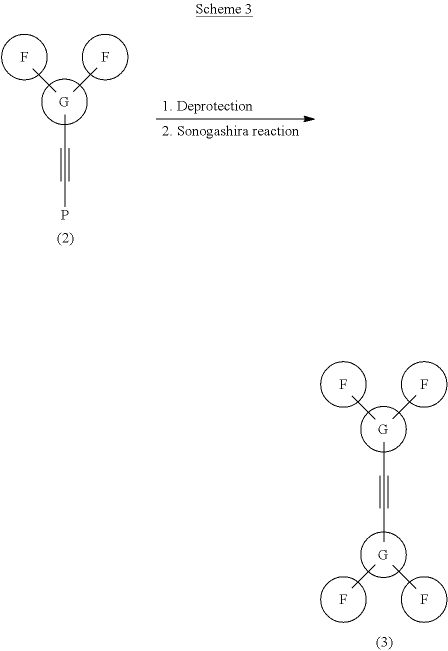

The F groups of Compound (3) can then be further functionalized to enable attachment of antigens. For example, acetyl groups can be introduced as shown in Scheme 4:

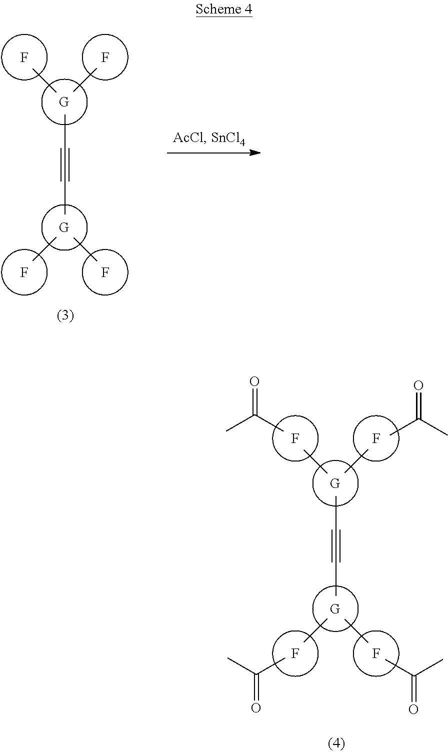

Compound (4) can undergo cyclization using a cobalt catalyst such as CO₂(CO)₈ and antigen incorporation in either order to produce a compound of formula (5) as shown in Scheme 5:

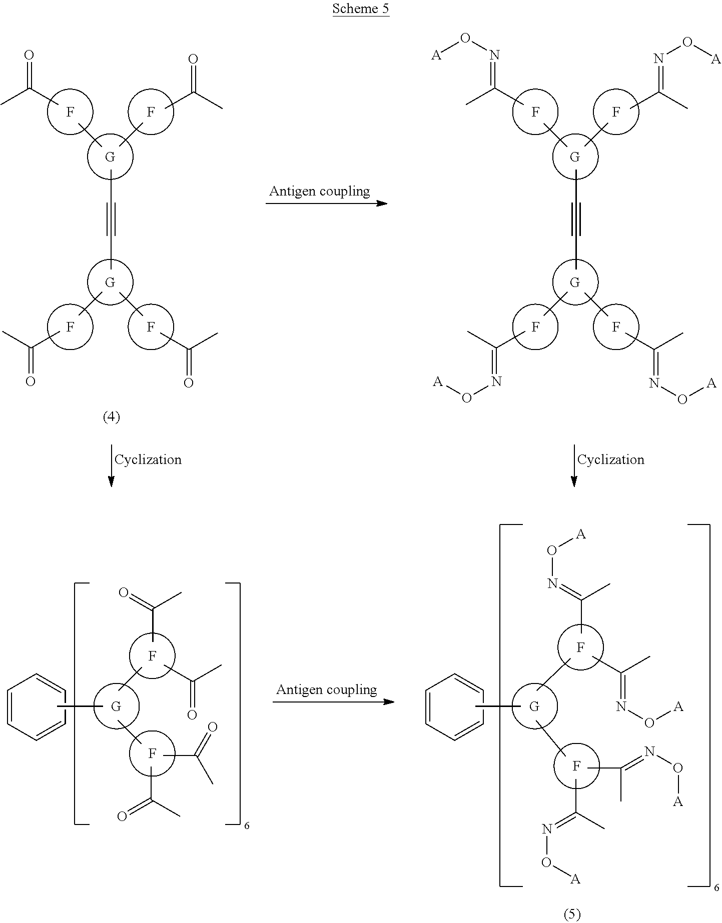

In Scheme 5, A represents an antigen. This pathway of Schemes 1 to 5 can be used to produce a compound in which all of the antigens presented on the Spacer, whether branched or unbranched, are the same.

In an alternative route for preparing a compound in which all of the antigens presented on the Spacer are the same, the F groups of Compound (1), whether one or two F groups are present, may be functionalized before the Sonogashira reactions to couple a first and a second Compound (1) to an acetylene group as shown in Scheme 6 (shown as disubstituted):

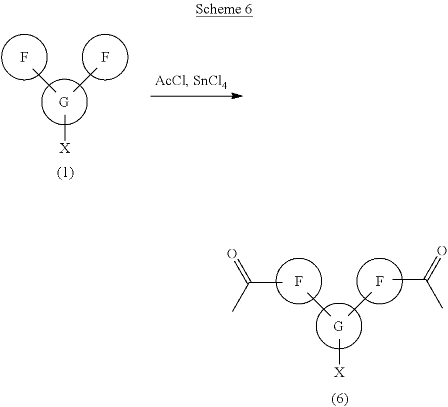

Compound 6 can then undergo a first Sonogashira reaction, deprotection, a second Sonogashira reaction, antigen coupling and cyclization as shown in Schemes 2, 3 and 5.

Similarly, if more than one antigen is to be attached to an F group, the amount of acetyl chloride can be increased as shown in Scheme 7 (shown as monosubstituted):

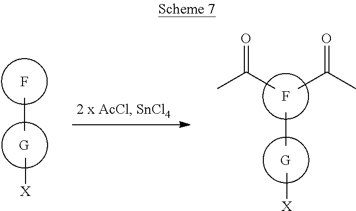

If it is required that the antigens presented on the Spacers are different, Compound (6) can be coupled with a first antigen in one reaction and a second antigen in a second reaction or Compound (6) could be reacted with a mixture of antigens. The product of coupling with the first antigen can then undergo Sonogashira coupling as shown in Scheme 8:

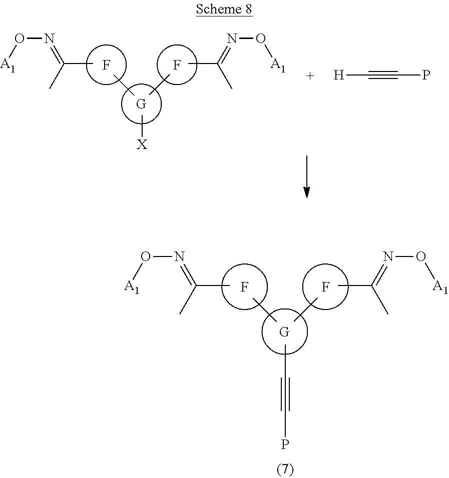

After deprotection of the acetylene group in Compound (7), a second Sonogashira reaction using a compound coupled to a different antigen is performed as shown in Scheme 9:

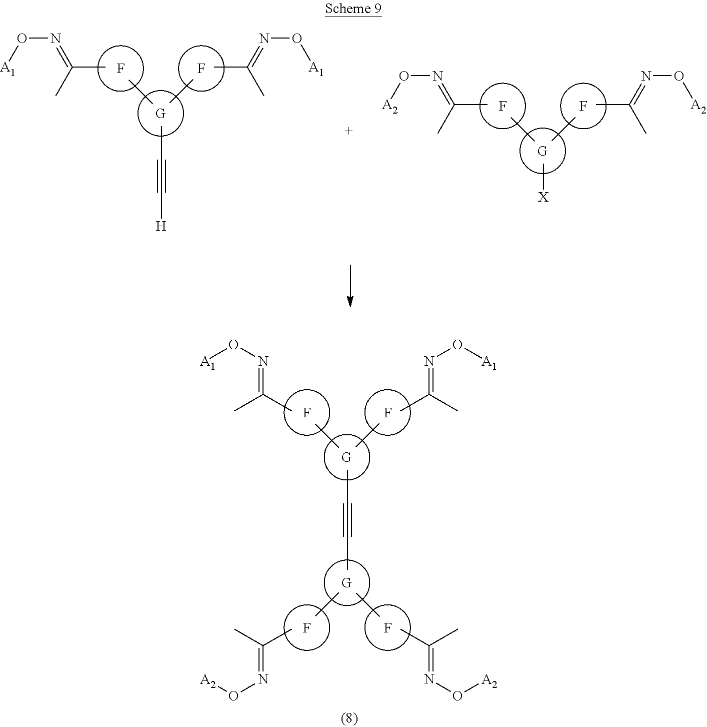

Finally, Compound 8 is cyclized with a catalyst such as a cobalt catalyst which may be CO₂(CO)₈, to provide a Compound of formula (I) presenting two different antigens as shown in Scheme 10.

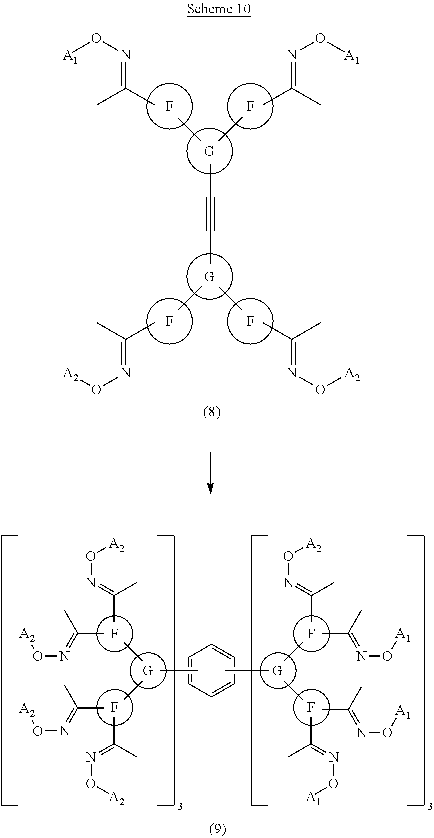

In other synthetic procedures, the compounds of the invention can be prepared using boronate ester derivatives. These reactions can be used to build an entire Spacer, either branched or unbranched or to attach a Spacer to a Core. For example, unbranched or branched Spacers may be prepared as shown in Scheme 11.

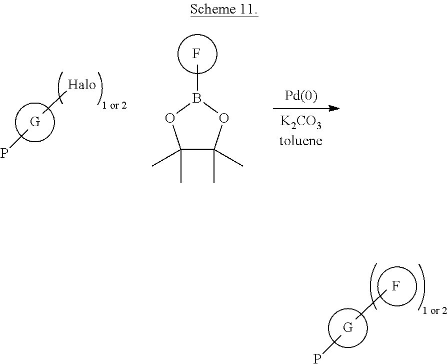

wherein P is another halo group or a bond to the Core or another aryl or heteroaryl group

Spacers, either branched or unbranched may also be attached to the Core in this manner, as shown in Scheme 12.

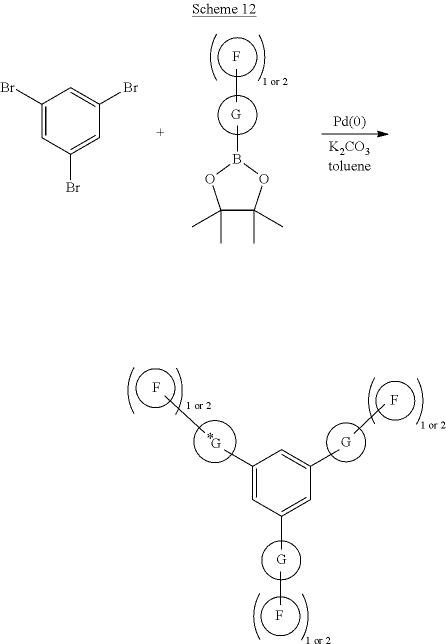

The antigen may be attached to the Spacer or incorporated into the DENDRITE by any method that utilizes functional groups available on the antigen and the Spacer or DENDRITE. For example, if the antigen contains an amino group available for coupling, the antigen may be coupled to the Spacer or into the DENDRITE by formation of an amide bond with a carboxy group on the DENDRITE moiety. If the antigen contains a hydroxy group or a carboxylic acid group, the antigen may be coupled to a DENDRITE moiety containing a corresponding carboxylic acid or hydroxy group to form an ester. In some embodiments, a functional group on the antigen may be further functionalized and reacted with the Spacer or DENDRITE moiety. For example, a hydroxy group on the antigen may be esterified with aminoxyacetic acid and then the amino group can be reacted with a carbonyl group, such as an aldehyde or ketone, present on the Spacer or DENDRITE moiety, to provide an oxime linkage.

Formation of amide bonds between the antigen and the Spacer or DENDRITE may be achieved using standard methods known in the art such as activation of a carboxylic acid and reaction with an amino group. Activation of the carboxylic acid may be achieved by formation of an acid chloride or anhydride or by use of coupling agents commonly used in peptide synthesis in the presence of base. Examples of suitable coupling agents include N—N′-carbonyldiimidazole (CDI), N,N′-dicyclohexylcarbodiimide (DCC), HBTU, benzyotriazole-1-yl-oxy-tris-(dimethylamino)-phosphonium hexafluorophosphate (BOP), 3-(Diethoxy-phosphoryloxy)-3H-benzo[d][1,2,3]-triazin-4-one (DEPBT), N,N′-diisopropylcarbodiimide (DIC), 1-ethyl-3-(3-dimethylaminopropyl)carbodiimide hydrochloride (EDC HCL), 2-(1H-2-Azabenzotriazol-1-yl)-1,1,3,3-tetramethyluronium hexafluorophosphate methanaminium (HATU), 1-hydroxy-7-azabenzotriazole (HOAt), N-hydroxybenzotriazole (HOBT), hydroxy-3,4-dihydro-4-oxo-1,2,3-benzotriazine (HOOBT), 1H-benzotriazolium-1-[bis(dimethylamino)methylene]-5-chloro-hexafluorophosphate-3-oxide (HCTU), 6-chloro-1-hydroxybenzotriazole (Cl—HOBt), benzotriazol-1-yl-oxytripyrrolidinophosphonium hexafluorophosphate (PyBOP), bromo-tris-pyrrolidinophosphonium hexafluorophosphate (PyBrOP), O-(benzotriazol-1-yl)-N,N,N′,N′-tetramethyluronium tetrafluoroborate (TBTU), N,N,N′,N′-tetramethyl-O-(3,4-dihydro-4-oxo-benzotriazin-3-yl)uronium tetrafluoroborate (TDBTU), 2-(7-aza-1H-benzotriazol-1-yl)-1,1,3,3-tetramethyluronium tetrafluoroborate (TATU), O—(N-succinimidyl)-1,1,3,3-tetramethyluronium tetrafluoroborate (TSTU) and 4,5-dicyanoimidazole.

Formation of ester bonds between the antigen and the Spacer or DENDRITE may be achieved using standard methods known in the art such as activation of a carboxylic acid and reaction with a hydroxy group. Activation of the carboxylic acid may be achieved by formation of an acid chloride or anhydride or use of a coupling agent as described for amide bond formation above.

Coupling of an aminooxy acetic acid linker group on the antigen with a carbonyl group on the dendrite to form oxime may be achieved by methods known in the art such as those described by Weikkolainen et al., Carbohydrate Polymers, 2007, 68:260-269. N-Boc-protected aminooxyacetic acid may be coupled to a hydroxy or amino group of the antigen in the presence of a coupling agent such as those described above for amide and ester bond formation. Exemplary conditions use the coupling agent HBTU in the presence of diisopropyl ethyl amine (DIPEA) in pyridine. After reaction is complete, the Boc-protecting group may be removed using trifluoroacetic acid (TFA). The amino group of the aminooxyacetyl group is then allowed to react with a carbonyl group on the Spacer or DENDRITE at pH 4.0.

Peptide antigens may be synthesized by methods known in the art such as solid phase synthesis, solution phase synthesis and recombinant techniques. In particular embodiments, the peptide antigens are synthesised by solid phase peptide synthetic techniques using Fmoc protected amino acids. The peptide is then coupled to the Spacer or DENDRITE by methods described above. For example, the N-terminus of the peptides is functionalized with Boc-protected aminooxyacetic acid which is commercially available. Upon cleavage of the peptide from the resin using TFA, the Boc protecting group is removed. This deprotected peptide is then reacted with a Spacer or DENDRITE carbonyl group to form an oxime in water or water/methanol at about pH 4 (sodium acetate) for about 12-15 hours.

Oligosaccharide antigens are synthesised using established carbohydrate synthetic techniques. The reducing end of the oligosaccharide is coupled to the Spacer or DENDRITE moiety as described above. For example, the reducing end of the oligosaccharide is functionalized with aminooxyacetic acid via an amide or ester linkage. This aminooxyacetic acid group will then be reacted with a carbonyl group of the Spacer or DENDRITE as described above.

Another method for attaching the a saccharide antigen to the Spacer or DENDRITE is using a 1,3-cycloaddition reaction where the anomeric hydroxy substituent of the saccharide is converted to an azide via a chloride and reacted with an acetylene group on the Spacer or DENDRITE thereby forming a 1,2,3-triazole attachment as shown in Scheme 13:

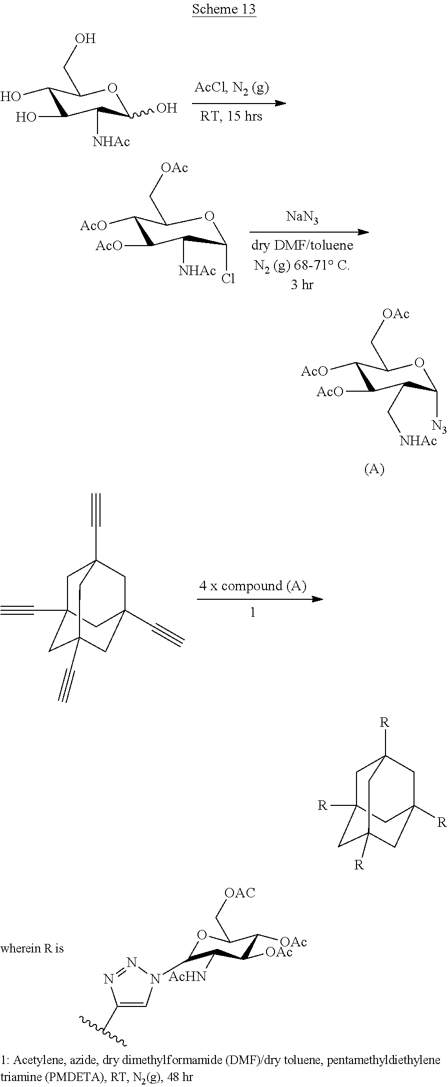

This 1,3-cycloaddition reaction may also be useful in linking an antigenic peptide with a Scaffold. For example, an antigenic peptide comprising a lysine residue in which the side chain amino group is derivatized to provide an azide can be reacted with a Scaffold comprising an acetylenyl group.

### Pharmaceutical Formulations

The present invention also contemplates immunomodulating formulations, including vaccines, which comprise the compounds broadly described above as active ingredients for modulating an immune response, e.g., for priming an immune response or for inducing a tolerogenic immune response to one or more cognate antigens. In some embodiments, the compounds of the present invention are useful for the treatment or prophylaxis of various diseases or conditions associated with the presence or aberrant expression of a target antigen. These therapeutic/prophylactic agents can be administered to a patient either by themselves or in formulations where they are mixed with a suitable pharmaceutically acceptable carrier and/or diluent, or an adjuvant.

The preparation of such formulations uses routine methods known to persons skilled in the art. Typically, such immunomodulating formulations and vaccines are prepared as injectables, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient. Suitable excipients are, for example, water, phosphate buffered saline, saline, dextrose, glycerol, ethanol, or the like and combinations thereof. In addition, if desired, the vaccine may contain minor amounts of auxiliary substances such as wetting or emulsifying agents, pH buffering agents, and/or adjuvants that enhance the effectiveness of the vaccine. Examples of adjuvants which may be effective include but are not limited to: surface active substances such as hexadecylamine, octadecylamine, octadecyl amino acid esters, lysolecithin, dimethyldioctadecylammonium bromide, N,N-dicoctadecyl-N′, N′ bis(2-hydroxyethyl-propanediamine), methoxyhexadecylglycerol, and pluronic polyols; polyamines such as pyran, dextransulfate, poly IC carbopol; mineral gels such as aluminum phosphate, aluminium hydroxide or alum; peptides such as muramyl dipeptide and derivatives such as N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 1983A, referred to as MTP-PE), and RIBI, which contains three components extracted from bacteria, monophosphoryl lipid A, dimethylglycine, tuftsin; oil emulions; trehalose dimycolate and cell wall skeleton (MPL+TDM+CWS) in a 2% squalene/Tween 80 emulsion; lymphokines; QuilA and immune stimulating complexes (ISCOMS). For example, the effectiveness of an adjuvant may be determined by measuring the amount of antibodies resulting from the administration of the immunomodulating formulation, wherein those antibodies are directed against one or more target antigens corresponding to the antigens presented by the compound of the invention.

The active ingredients should be administered in a pharmaceutically acceptable carrier, which is non-toxic to the cells and the individual to be treated. Such carrier may be the growth medium in which the cells were grown. Compatible excipients include isotonic saline, with or without a physiologically compatible buffer like phosphate or Hepes and nutrients such as dextrose, physiologically compatible ions, or amino acids, and various culture media suitable for use with cell populations, particularly those devoid of other immunogenic components. Carrying reagents, such as albumin and blood plasma fractions and nonactive thickening agents, may also be used. Non-active biological components, to the extent that they are present in the formulation, are preferably derived from a syngeneic animal or human as that to be treated, and are even more preferably obtained previously from the subject. The injection site may be subcutaneous, intraperitoneal, intramuscular, intradermal, or intravenous.

If soluble actives are employed, the soluble active ingredients can be formulated into the formulation as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric, maleic, and the like. Salts formed with the free carboxyl groups may also be derived from inorganic basis such as, for example, sodium, potassium, ammonium, calcium, or ferric hydroxides, and such organic bases as isopropylamine, trimethylamine, 2-ethylamino ethanol, histidine, procaine, and the like.

If desired, devices or pharmaceutical compositions or compositions containing the compounds of the invention and suitable for sustained or intermittent release could be, in effect, implanted in the body or topically applied thereto for the relatively slow release of such materials into the body.

Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

The dosage to be administered may depend on the subject to be treated inclusive of the age, sex, weight and general health condition thereof. The dosage will also take into consideration the binding affinity of the lectin-interactive agent to the lectins, its bioavailability and its in vivo and pharmacokinetic properties. In this regard, precise amounts of the agent(s) for administration can also depend on the judgement of the practitioner. In determining the effective amount of the agent(s) to be administered in the treatment of a disease or condition, the physician or veterinarian may evaluate the progression of the disease or condition over time. In any event, those of skill in the art may readily determine suitable dosages of the agents of the invention without undue experimentation. The dosage of the actives administered to a patient should be sufficient to achieve a beneficial response in the patient over time such as a reduction in the symptoms associated with the disease or condition to be treated. For example usual patient dosages for systemic administration of carbohydrate lectin-interactive agents range from 0.1-200 g/day, typically from 1-160 g/day and more typically from 10-70 g/day. Stated in terms of patient body weight, usual dosages range from 1.5-3000 mg/kg/day, typically from 15-2500 mg/kg/day and more typically from 150-1000 mg/kg/day.

In some embodiments the pharmaceutical formulations further comprises one or more ancillary agents such as but not limited to cytokines, which are suitably selected from flt3, SCF, IL-3, IL-6, GM-CSF, G-CSF, TNF-α, IL-4, TNF-β, LT-β, IL-2, IL-7, IL-9, IL-15, IL-13, IL-5, IL-1α, IL-1β, IFN-γ, IL-10, IL-17, IL-16, IL-18, HGF, IL-11, MSP, FasL, TRAIL, TRANCE, LIGHT, TWEAK, CD27L, CD30L, CD40L, APRIL, TALL-1, 4-1BBL, OX40L, GITRL, IGF-I, IGF-II, HGF, MSP, FGF-a, FGF-b, FGF-3-19, NGF, BDNF, NTs, Tpo, Epo, Angl-4, PDGF-AA, PDGF-BB, VEGF-A, VEGF-B, VEGF-C, VEGF-D, P1GF, EGF, TGF-α, AR, BTC, HRGs, HB-EGF, SMDF, OB, CT-1, CNTF, OSM, SCF, Flt-3L, M-CSF, MK and PTN or their functional, recombinant or chemical equivalents or homologues thereof. In some embodiments, the cytokine is selected from the group consisting of IL-12, IL-3, IL-5, TNF, GMCSF, and IFN-γ.

### Methods for Modulating Immune Responses

The compositions of the invention may be used for modulating an immune response in a subject. Thus, in one embodiment there is provided a method for enhancing an immune response in a subject by administering to the subject the compounds or compositions of the invention. In some embodiments, the immune response is a humoral immune response (e.g., a B-cell mediated response, which desirably includes CD4+ T cells); in others it is a cell-mediated immune response (e.g., a T-cell mediated response, which desirably includes CD4+ and/or CD8+T cells).

The active ingredients of the compositions may be administered sequentially, simultaneously or separately.

Also encapsulated by the present invention are methods for treatment and/or prophylaxis of a disease or condition, comprising administering to a patient in need of such treatment an effective amount of a compound or composition as broadly described above. In certain embodiments, the compound or composition is designed to stimulate or augment an immune response to a target antigen. In these embodiments, the target antigen is typically associated with or responsible for a disease or condition which is suitably selected from cancers, infectious diseases and diseases characterised by immunodeficiency. Examples of cancer include but are not limited to ABL1 protooncogene, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, dermatofibrosarcoma-protuberans, desmoplastic-small-round-cell-tumour, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal-carcinoid-tumour, genitourinary cancers, germ cell tumours, gestational-trophoblastic-disease, glioma, gynaecological cancers, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukaemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer-(NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, rare-cancers-and-associated-disorders, renal cell carcinoma, retinoblastoma, rhabdomyosarcoma, Rothmund-thomson syndrome, salivary gland cancer, sarcoma, schwannoma, Sezary syndrome, skin cancer, small cell lung cancer (SCLC), small intestine cancer, soft tissue sarcoma, spinal cord tumours, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplalcins, uterine sarcoma, uterus cancer, vaginal cancer, vulva cancer, Waldenstrom's-macroglobulinemia, Wilms' tumour.

In other embodiments, the compound or composition of the invention are used for generating large numbers of CD8⁺ or CD4+ T lymphocytes, for adoptive transfer to immunodeficient individuals who are unable to mount normal immune responses. For example, antigen-specific CD8⁺ T lymphocytes can be adoptively transferred for therapeutic purposes in individuals afflicted with HIV infection (Koup et al., 1991, J. Exp. Med. 174: 1593-1600; Carmichael et al., 1993, J. Exp. Med. 177: 249-256; and Johnson et al., 1992, J. Exp. Med. 175: 961-971), malaria (Hill et al., 1992, Nature 360: 434-439) and malignant tumours such as melanoma (Van der Brogen et al., 1991, Science 254: 1643-1647; and Young and Steinman 1990, J. Exp. Med., 171: 1315-1332).

In other embodiments, the compound or composition is suitable for treatment or prophylaxis of a viral, bacterial or parasitic infection. Viral infections contemplated by the present invention include, but are not restricted to, infections caused by HIV, Hepatitis, Influenza, Japanese encephalitis virus, Epstein-Barr virus and respiratory syncytial virus.

Bacterial infections include, but are not restricted to, those caused by Neisseria species, Meningococcal species, Haemophilus species Salmonella species, Streptococcal species, Legionella species and Mycobacterium species. Parasitic infections encompassed by the invention include, but are not restricted to, those caused by Plasmodium species, Schistosoma species, Leishmania species, Trypanosoma species, Toxoplasma species and Giardia species.

In still other embodiments, the compound or composition of the present invention is designed to induce tolerance or otherwise attenuate an immune response to a target antigen. In these embodiments, the target antigen is typically associated with or responsible for a disease or condition which is suitably selected from transplant rejection, graft versus host disease, allergies, parasitic diseases, inflammatory diseases and autoimmune diseases. Examples of transplant rejection, which can be treated or prevented in accordance with the present invention, include rejections associated with transplantations bone marrow and of organs such as heart, liver, pancreas, kidney, lung, eye, skin etc. Examples of allergies include asthma, hayfever, food allergies, animal allergies, atopic dermatitis, rhinitis, allergies to insects, fish, latex allergies etc. Autoimmune diseases that can be treated or prevented by the present invention include, for example, psoriasis, systemic lupus erythematosus, myasthenia gravis, stiff-man syndrome, thyroiditis, Sydenham chorea, rheumatoid arthritis, diabetes and multiple sclerosis. Examples of inflammatory disease include Crohn's disease, colitis, chronic inflammatory eye diseases, chronic inflammatory lung diseases and chronic inflammatory liver diseases.

The effectiveness of the immunization or tolerance may be assessed using any suitable technique. For example, CTL lysis assays may be employed using stimulated splenocytes or peripheral blood mononuclear cells (PBMC) on peptide coated or recombinant virus infected cells using ⁵¹Cr or Alamar Blue™ labeled target cells. Such assays can be performed using for example primate, mouse or human cells (Allen et al., 2000, J. Immunol. 164(9): 4968-4978 also Woodberry et al., infra). Alternatively, the efficacy of the immunization may be monitored using one or more techniques including, but not limited to, HLA class I tetramer staining—of both fresh and stimulated PBMCs (see for example Allen et al., supra), proliferation assays (Allen et al., supra), ELISPOT assays and intracellular IFN-γ staining (Allen et al., supra), ELISA Assays—for linear B cell responses; and Western blots of cell sample expressing the synthetic polynucleotides

### EXAMPLES

### Example 1

### Synthesis of Compound (10)

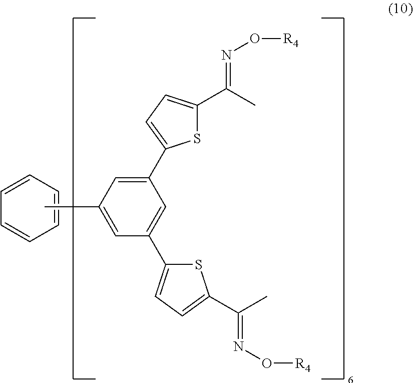

where R₄ is —CH₂CO₂N—PIA disaccharide.

Two equivalents of 2-thiophenyl(tributyl)stannane and 1,3,5-tribromobenzene are subject to Stille Coupling conditions using a Pd(0) catalyst to produce 1-bromo-3,5-dithienylbenzene. The 1-bromo-3,5-dithienylbenzene is reacted with 3,3-dimethylpropargyl-3-ol under Sonogashira coupling conditions in the presence of PdCl₂.(PPh₃)₂, CuI, Et₃N at room temperature, followed by deprotection of the alkyne protecting group with aqueous sodium hydroxide in benzene under reflux conditions to produce 1-ethynyl-3,5-dithienylbenzene. 1-ethynyl-3,5-dithienylbenzene is then subjected to a second Sonogashira coupling to produce 1,2-(di-1-[3,5-dithienylphenyl])ethyne. Functionalization of the thienyl groups in the 5 position is achieved by treating 1,2-(di-1-[3,5-dithienylphenyl])ethyne with 4 equivalents of acetyl chloride in the presence of tin chloride (SnCl₄). The acetyl carbonyl groups were then reacted with an hydroxy group of a PIA disaccharide antigen by initially substituting the hydroxy group of the disaccharide with an amine by treatment with HBr/HOAc and NaN₃ followed by reduction of the azide with H₂/Pd/C. The amino group of the disaccharide is then substituted with N-Boc protected aminooxyacetic acid in the presence of DCC followed by Boc deprotection with TFA. This was followed by treatment with sodium ethoxide and ethanol, with Dowex H⁺ resin. The aminooxyacetic amide disaccharide antigen is then reacted with the acetyl carbonyl group of the substituted thienyl moiety. Finally three equivalents of the antigen coupled compound were cyclized with CO₂(CO)₈ to provide compound (10).

### Example 2

### Preparation of Compound 11

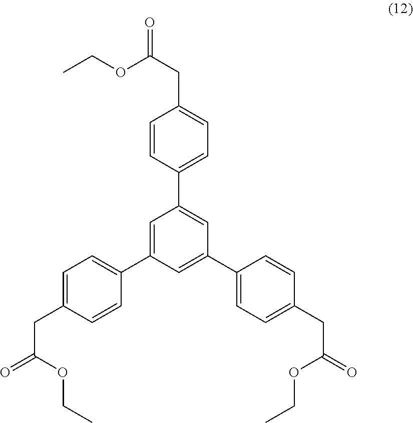

To a 50 mL pressure tube was added 4-(ethoxycarbonylmethyl)phenylboronic acid, pinacol ester (0.325 grams, 0.0011 mole), 1,3,5-tribromobenzene (0.1 grams, 0.00032 mole), dioxane (30 mL) and potassium phosphate tribasic (2.44 grams, 0.01150 mole), and was purged with nitrogen gas for 15 min. To this solution was added tetrakis(triphenylphosphine)palladium(0) (0.09 grams, 0.000008 mole) and degassed and purged with nitrogen gas three times. The flask was sealed and heated to 90° C. with gentle stirring for 48 hours. Dichloromethane (50 mL) and water (100 mL) were added. The organic layer was separated, and the aqueous layer was extracted with dichloromethane (2×50 mL). The combined organic layers were washed with water (2×100 mL) and brine (100 mL), dried over anhydrous magnesium sulfate, and filtered, and then the solvent was removed. The residue was purified by column chromatography (hexane:ethyl acetate gradient) to result in [12] (0.125 grams, ˜70% yield). ¹H NMR (500 MHz, CDCl₃, PPM) δ=7.74 (s, 3H), 7.64 (d, j=8 Hz, 6H), 7.40 (d, j=8 Hz, 6H), 4.20 (q, j=19.2 & 19.7, 6H), 3.67 (s, 6H), 1.29 (t, j=19.3, 9H). m/z [TOF EST] 587 (M⁺+Na).

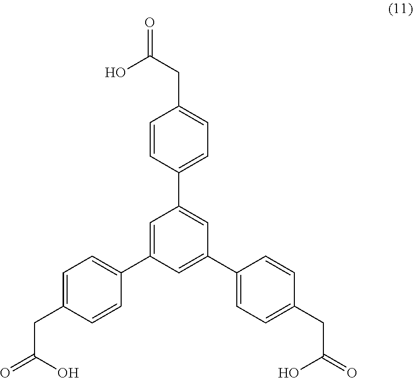

The tri-ester [12] (96.4 mg, 1.71×10⁴ mol) was dissolved in a mixture of EtOH/H₂O (3:1, 12 mL), cooled to ˜5° C. and NaOH (0.2 g, 5.0 mmol) was added. The reaction mixture was then stirred at room temperature for 4 h. Ethanol was evaporated under reduced pressure and the aqueous mixture was acidified with HCl (2M). The white solid obtained was filtered, washed with H₂O and dried under high vacuum to give [11] as a white solid (80.4 mg, 98%). EIMS: M⁺=480.15. ¹Hnmr (500 MHz, CDCl₃/Acetone-d₆) δ 7.45, s, 3H, Ar—H, 7.35, d, 611, J=8.2 Hz, Ar—H, 7.11, d, 6H, J=8.2 Hz, Ar—H, 3.38, s, 6H, Ph-CH₂—CO₂H. ¹³C nmr (125 MHz, CDCl₃/Acetone-d₆) δ 172.17 (—CO₂H); 141.41; 139.09; 133.35; 129.33; 126.76; 124.18; 40.00 (Ph-CH₂—CO₂H).

### Example 3

### Preparation of Compound (13)

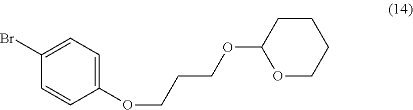

To a 500 mL round bottom flask was added 4-bromophenol (7.65 g, 0.044 mole) and 80 mL of acetonitrile. To this solution was added potassium tert-butoxide (4.6 grams, 0.041 mole) portionwise over 15 min, followed by refluxing for 1 hour. The solution was cooled to room temperature and 2-(3-bromopropoxy)tetrahydro-2H-pyran (8.3 grams, 0.037 mole) was added. This solution was refluxed for 24-48 hours. Dichloromethane (100 mL) and water (150 mL) were added. The organic layer was separated, and the aqueous layer was extracted with dichloromethane (3×100 mL). The combined organic layers were washed with water (2×100 mL) and brine (100 mL), dried over anhydrous magnesium sulfate, and filtered, and then the solvent was removed. The residue was purified by silica gel chromatography using a gradient solvent hexane:ethyl acetate system to result in [14] as a colourless oil (8.8 grams, ˜76% yield). ¹H NMR (300 MHz, CDCl₃, PPM) δ 7.38 (m, 2H), 6.80 (m, 2H), 4.60 (t, j=12.8 Hz, 1H), 4.05 (t, j=6.3 Hz, 2H), 3.96-3.81 (m, 2H), 3.61-3.46 (m, 2H), 2.07 (quintet, j=6.2 Hz, 2H), 1.80-1.4 (m, 6H). m/z [TOF ESI] 337 (MH⁺).

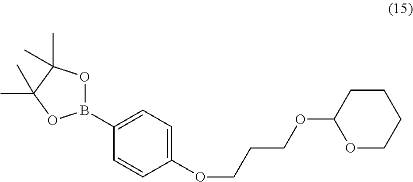

To a dry 50 mL round bottom flask was added [14] (3.8 grams, 0.012 mole) and 25 mL of freshly distilled tetrahydrofuran, and cooled to −78 C. To this solution was added 8.6 mL of 1.6 M n-butyllithium (13.8 mmol) over 30 min. After stirring this solution for 1.5 hours at −78° C., 2-isopropoxy-4,4,5,5-tetramethyl-1,3,2-dioxaborolane (2.54 grams, 0.0138 mole) was added. This solution was left to stir and warm up to room temperature overnight. The solvent was removed under reduced pressure. Dichloromethane (50 mL) and water (100 mL) were added. The organic layer was separated, and the aqueous layer was extracted with dichloromethane (2×50 mL). The combined organic layers were washed with water (2×100 mL) and brine (100 mL), dried over anhydrous magnesium sulfate, and filtered, and then the solvent was removed and resulted in a light yellow oil that contained [15] (3.52 grams, ˜81% yield). ¹H NMR (300 MHz, CDCl₃, PPM) δ 7.74-7.71 (m, 2H), 6.90-6.87 (m, 2H), 4.59 (t, j=4.2 Hz, 1H), 4.10 (t, j=6.4 Hz, 2H), 3.95-3.80 (m, 2H), 3.60-3.40 (m, 2H), 2.07 (quintet, j=6.2 Hz, 2H), 1.48-1.46 (m, 6H). 1.32 (s, 12H). m/z [TOF ESI] 386 (M⁺+Na).

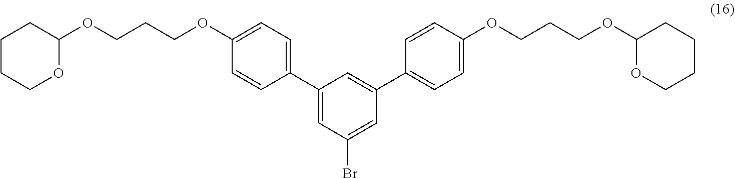

To 100 mL pressure tube was added [15] (4 grams, 0.011 mole), 1,3,5-tribromobenzene (1.54 grams, 0.049 mole), 40 mL of toluene and 2M K₂CO₃ (4 mL). This solution was purged for 5 min with nitrogen gas. Then tetrakis(triphenylphosphine)palladium(0) (0.170 grams, 0.000147 mole) was added and the solution degassed and purged three times with nitrogen gas. This solution was stirred rapidly for 60 hours at 100° C. Dichloromethane (100 mL) and water (150 mL) were added. The organic layer was separated, and the aqueous layer was extracted with dichloromethane (2×100 mL). The combined organic layers were washed with water (1×100 mL) and brine (100 mL), dried over anhydrous magnesium sulfate, and filtered, and then the solvent was removed. The residue was purified by silica gel chromatography using a gradient solvent hexane:ethylacetate system from 0% ethyl acetate to 50% ethyl acetate resulting in [16] as a colourless oil (2.0 grams, ˜65% yield). ¹H NMR (300 MHz, CDCl₃, PPM) δ 7.60 (m, 3H), 7.53-7.50 (m, 4H), 6.70-6.96 (m, 4H), 4.61 (m, 2H), 4.13 (t, j=6.5 Hz, 4H), 4.0-3.8 (m, 4H), 3.63-3.46 (m, 4H), 2.10 (quintet, j=6.3, 4H), 1.87-1.47 (m, 12H). m/z [TOF ESI] 649 (M++Na).

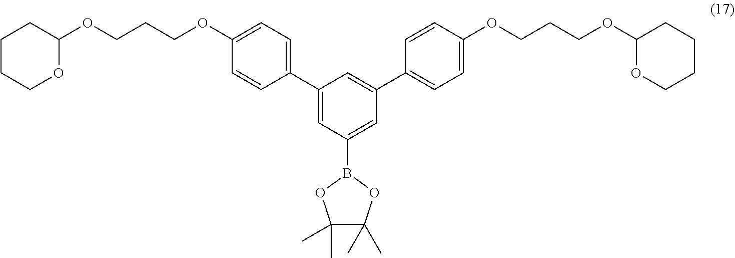

To a 100 mL round bottom flask was added [16] (1.45 g, 0.00232 mole) and dry tetrahydrofuran (10 mL). This solution was cooled to −78° C. and 1.7 mL of n-BuLi was added dropwise over 15 min. This solution was left to stir for 1.5 hours at −78° C. and 2-isopropoxy-4,4,5,5-tetramethyl-1,3,2-dioxaborolane (0.52 g, 0.00278 mole) was added. This solution was left to slowly warm up to room temperature overnight. The solvent was removed under reduced pressure. Dichloromethane (50 mL) and water (50 mL) were added. The organic layer was separated, and the aqueous layer was extracted with dichloromethane (2×50 mL). The combined organic layers were washed with water (2×50 mL) and brine (50 mL), dried over anhydrous magnesium sulfate, and filtered, and then the solvent was removed to contain [17] as a viscous yellow oil (1.22 grams, ˜78% yield). This compound could be purified by using a chromatotron using a gradient solvent system with hexane and diethyl ether. ¹H NMR (300 MHz, CDCl₃, PPM) δ 7.93 (d, j=1.8 Hz, 2H), 7.80 (t, j=1.9 Hz, 1H), 7.60-7.57 (m, 4H), 6.98-6.95 (m, 4H), 4.62-4.60 (m, 2H), 4.13 (t, j=6.5 Hz, 4H), 3.98-3.82 (m, 4H), 3.64-3.47 (m, 4H), 2.10 (quintet, j=6.25 Hz, 4H), 1.93-1.48 (m, 12H), 1.36 (s, 12H). m/z [TOF ESI] 672 (MH⁺).

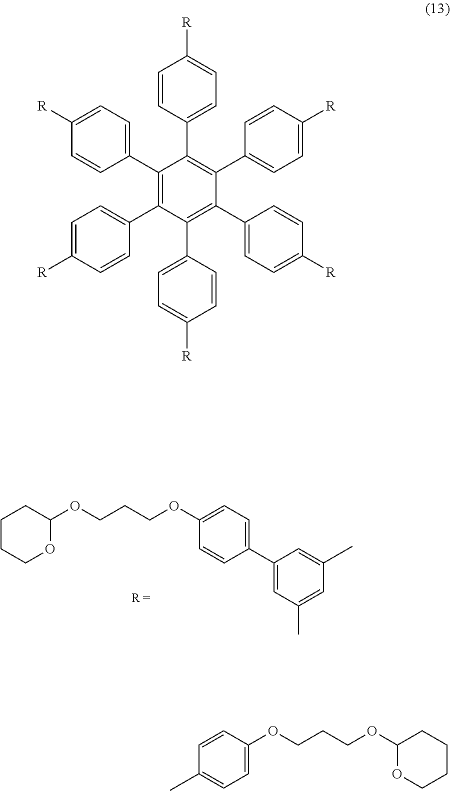

To a 50 mL pressure tube was added hexakis(4-iodophenyl)benzene (0.063 grams, 0.00005 mole), [17] (0.330 grams, 0.00049 mole) and 20% tetraethylammonium hydroxide (5 mL). This solution was purged with nitrogen gas for 45 min. To this solution was added tetrakis(triphenylphosphine)palladium(0) (0.035 grams, 0.00003 mole) and the solution was degassed-purged three times with nitrogen. The pressure tube was sealed under nitrogen and heated to 100° C. for 60 hours. Dichloromethane (50 mL) and water (100 mL) were added. The organic layer was separated, and the aqueous layer was extracted with dichloromethane (2×50 mL). The combined organic layers were washed with water (2×50 mL) and brine (50 mL), dried over anhydrous magnesium sulfate, filtered, and then the solvent was completely removed. The residue was passed though a celite plug with diethyl ether as the eluent, the solvent was removed. The residue was purified with 50 grams of LH-20 Sephadex size exclusion chromatography using tetrahydrofuran as the eluent and a chromatotron using a gradient of dichloromethane and ethyl acetate, resulting in [13] as a white powder (0.130 grams, ˜56% yield). ¹H NMR (300 MHz, CDCl₃, PPM) δ 7.54-7.52 (m, 18H), 7.48-7.46 (m, 24H), 7.32-7.30 (m, 12H), 7.05-7.03 (m, 12H), 6.88-6.86 (m, 24H), 4.59-4.57 (m, 12H), 4.07-4.02 (m, 24H), 3.93-3.80 (m, 24H), 3.59-3.45 (m, 24H), 2.09-2.02 (m, 24H), 1.84-1.65 (m, 24H), 1.59-1.46 (m, 48H) GPC: MP 3875, PDI 1.00.

### Example 4

### Preparation of Compound (18)

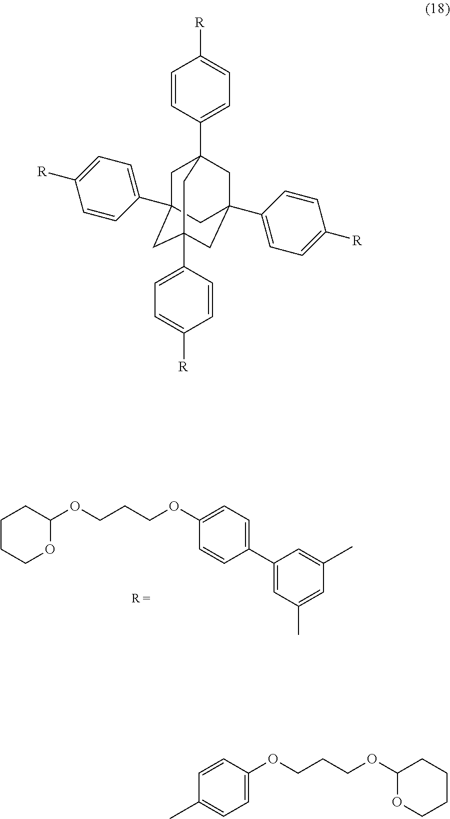

To a 100 mL round bottom flask was added [17] (0.450 g, 0.00067 mole) as prepared in Example 3, tetrakis(4-iodophenyl)-1,1′,1″,1′″-adamantane (0.098 grams, 0.000104 mole), toluene (10 mL) and 20% tetraethyl ammonium hydroxide (5 mL). This solution was degassed with nitrogen gas for 15 min. To this solution was added tetrakis(triphenylphosphine)palladium(0) (0.045 grams, 0.00004 mole) and the solution was purged-degassed with nitrogen three times and reacted under a nitrogen atmosphere for 60 hours. Dichloromethane (50 mL) and water (50 mL) were added. The organic layer was separated, and the aqueous layer was extracted with dichloromethane (2×50 mL). The combined organic layers were washed with water (2×50 mL) and brine (50 mL), dried over anhydrous magnesium sulfate, filtered, and then the solvent was completely removed. The crude ¹H NMR showed a broad singlet at 2.35 ppm from the adamantyl group, indicating that the compound was obtained. The residue was purified with 50 grams of LH-20 Sephadex size exclusion chromatography using tetrahydrofuran as the eluent, resulting in a light brown oil containing (18). ¹H NMR (300 MHz, CDCl₃, ppm) δ 7.74-7.61 (m, 44H), 7.03, 7.01 (m, 4H), 4.6 (m, 8H), 4.18-3.50 (m), 2.36 (s, 12H), 2.20-1.50 (m).

### Example 5

### Preparation of Compound (19)

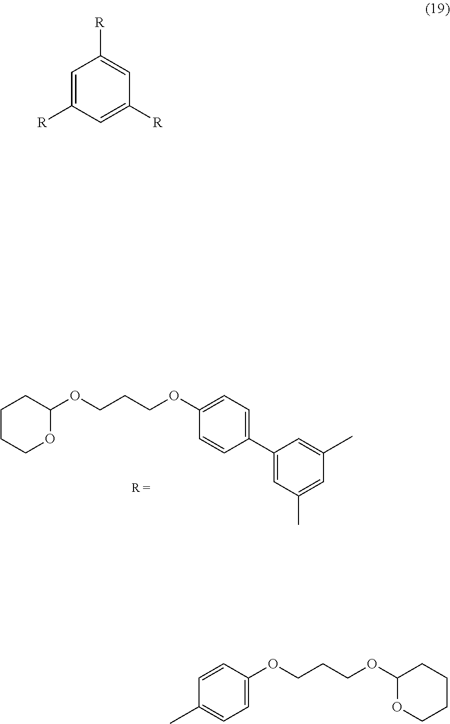

To a 100 mL round bottom flask was added [17] (0.320 g, 0.00048 mole) as prepared in Example 3, 1,3,5-tribromobenzene (0.029 g, 0.00009 mole), toluene (10 mL) and 20% tetraethyl ammonium hydroxide (5 mL). This solution was degassed with nitrogen gas for 15 min. To this solution was added 30 mg of tetrakis(triphenylphosphine)palladium(0), the solution was purged-degassed with nitrogen three times and reacted under a nitrogen atmosphere for 48 hours. Dichloromethane (50 mL) and water (100 mL) were added. The organic layer was separated, and the aqueous layer was extracted with dichloromethane (2×50 mL). The combined organic layers were washed with water (2×50 mL) and brine (50 mL), dried over anhydrous magnesium sulfate, filtered, and then the solvent was removed. The residue was purified using a chromatotron (hexane:ethyl acetate gradient) to result in a viscous oil containing [19]. ¹H NMR (500 MHz, CDCl₃, PPM) δ=7.96 (s, 3H), 7.80 (d, j=1.6 Hz, 6H), 7.74 (t, j=1.6 Hz, 3H), 7.63-7.62 (m, 12H), 7.0-6.99 (m, 12H), 4.61-4.60 (m, 6H), 4.15-4.11 (m, 12H), 3.96-3.92 (m, 6H), 3.87-3.82 (m, 6H), 3.61-3.57 (m, 6H), 3.51-3.47 (m, 6H), 1.84-1.77 (m, 6H), 1.73-1.68 (m, 6H), 1.60-1.47 (m, 36H).

### Example 6

### Preparation of Compound (20)

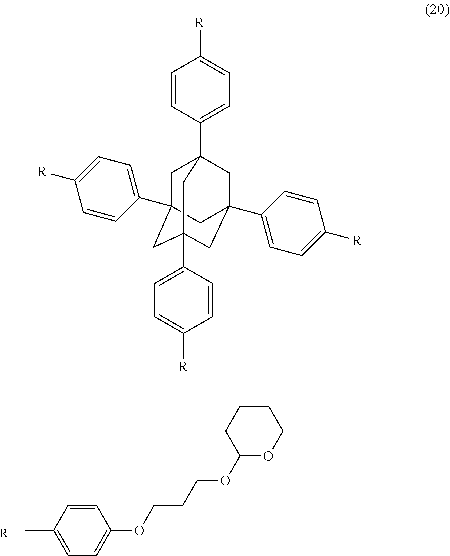

To a 50 mL schlenk flask was added tetrakis(4-iodophenyl)-1,1′,1″,1′″-adamantane (0.1 gram, 0.000106 mole), [15] (0.24 gram, 0.000635 mole) as prepared in Example 3, 20 mL of toluene and 2 mL of 2M K₂CO₃ (aq). This solution was purged with nitrogen gas for 15 min. To this solution was added tetrakis(triphenylphosphine)palladium(0) (0.025 g, 0.000019 mole) and the solution was degassed and purged with nitrogen three times. This solution was heated to 90° C. for 24 hours under vigorous stirring. Dichloromethane (50 mL) and water (50 mL) were added. The organic layer was separated, and the aqueous layer was extracted with dichloromethane (2×50 mL). The combined organic layers were washed with water (2×50 mL) and brine (50 mL), dried over anhydrous magnesium sulfate, and filtered, and then the solvent was completely removed to result in a viscous brown oil. This oil was passed through a celite plug with diethyl ether, the solvent was completely removed and the residue was purified by silica gel chromatography using a gradient solvent hexane:ethylacetate system from 0% ethyl acetate to 50% ethyl acetate resulting in [20] as a white powder (0.020 grams, ˜14% yield). ¹H NMR (500 MHz, CDCl₃, PPM) δ 7.58-7.51 (m, 24H), 6.98-6.96 (m, 8H), 4.61-4.60 (m, 4H), 4.15-4.09 (m, 8H), 3.96-3.92 (m, 4H), 3.87-3.83 (m, 4H), 3.62-3.58 (m, 4H), 3.52-3.48 (m, 4H), 2.26 (s, 12H), 2.12-2.06 (m, 8H), 1.86-1.85 (m, 4H), 1.74-1.68 (m, 4H), 1.60-1.48 (m, 16H). GPC: Mp=1855, PDI=1.00.

### Example 7

### Preparation of Compound (21)

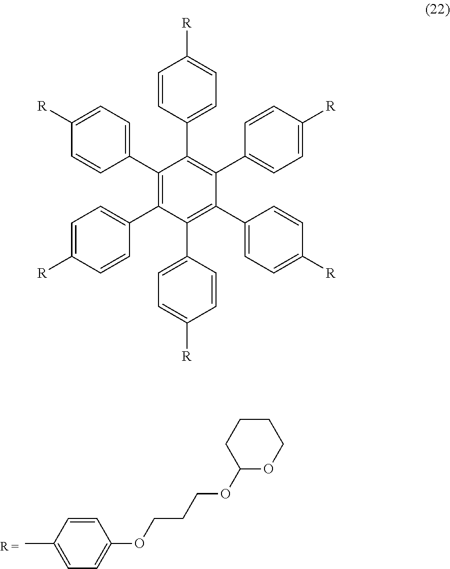

To a 100 mL pressure tube was added hexakis(4-iodophenyl)benzene (0.20 grams, 0.0001551 mole), [15] (0.674 grams, 0.00186 mole) as prepared in Example 3, toluene (25 mL), 35% tetraethyl ammonium hydroxide (5 mL) and water (2.5 mL). This solution was heated to 100° C. for 24 hours. Dichloromethane (50 mL) and water (100 mL) were added. The organic layer was separated, and the aqueous layer was extracted with dichloromethane (2×50 mL). The combined organic layers were washed with water (2×100 mL) and brine (100 mL), dried over anhydrous magnesium sulfate, and filtered, and then the solvent was completely removed. The residue was purified by silica gel chromatography using a gradient solvent hexane:ethylacetate system. The residue was recrystallized from a mixed solvent system containing dichloromethane and hexane resulting in [22] as a colourless film (0.140 grams, ˜47% yield). ¹H NMR (500 MHz, CDCl₃, PPM) δ 7.34-7.32 (m, 12H), 7.10-7.08 (m, 12H), 6.91-6.89 (m, 12H), 6.85-6.83 (m, 12H), 4.58-5.57 (m, 6H), 4.08-4.02 (m, 12H), 3.92-3.87 (m, 6H), 3.84-3.80 (m, 6H), 3.57-3.53 (m, 6H), 3.49-3.45 (m, 6H), 2.07-2.02 (m, 12H), 1.82-136 (m, 6H), 1.71-1.65 (m, 6H), 1.57-1.46 (m, 24H) ink [TOP ESI] 1962 (M⁺+Na).

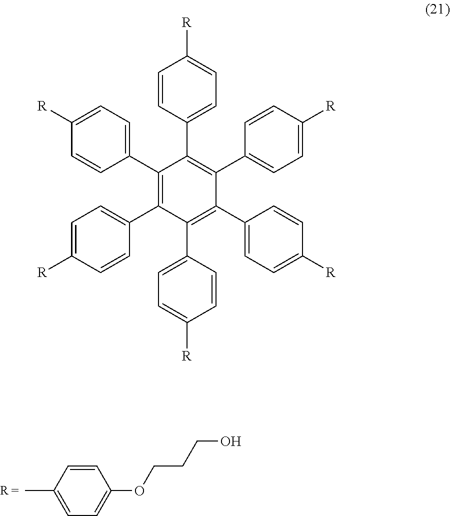

A solution of the tetrahydropyran (THP) protected alcohol (22) (53 mg, 2.73×10⁻⁵ mol) in THF/MeOH (2:1, 3 mL) was stirred with HCl (32%, 4 drops) at room temperature for 20 h. The solvent was removed in vacuo and the residue was dried under high vacuum to give the alcohol as a white solid (39.2 mg, 100%). ¹Hnmr (500 MHz, CDCl₃/CD₃OD (3 drops)) δ 7.21, d, 12H, J=8.8 Hz, Ar—H, 7.00, d, 12H, J=8.4 Hz, Ar—H, 6.86, d, 12H, J=8.4 Hz, Ar—H, 6.72, d, 12H, J=8.8 Hz, Ar—H, 3.96, t, 12H, J=6.2 Hz, Ph-O—CH₂—CH₂—CH₂—OH; 3.68, t, 12H, J=6.2 Hz, Ph-O—CH₂—CH₂—CH₂—OH, 1.90, m, 12H, Ph-O—CH₂—CH₂—CH₂—OH. ¹³C nmr (125 MHz, CDCl₃) δ 157.90 (Ar—C); 140.14 (Ar—C); 139.05 (Ar—C); 136.86

(Ar—C); 133.17 (Ar—C); 131.76 (Ar—CH); 127.49 (Ar—CH); 124.61 (Ar—CH); 114.39 (Ar—CH); 65.06 (Ph-O—CH₂—CH₂—CH₂—OH); 59.15 (Ph-O—CH₂—CH₂—CH₂—OH); 31.78 (Ph-O—CH₂—CH₂—CH₂—OH).

### Example 8

### Preparation of Compound (23)

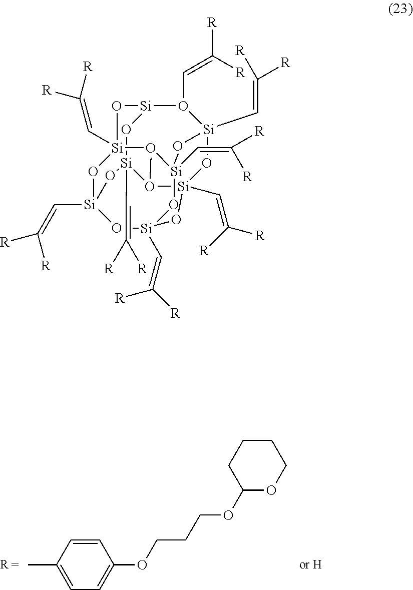

To a 100 mL pressure tube was added octavinylsilsesquioxane (0.050 g, 0.000079 mole), [14] (0.59 grams, 0.0019 mole) as prepared in Example 3, and toluene (14 mL). This solution was purged with nitrogen gas for 20 min. To this solution was added bis-(tri-tert-butylphosphine) palladium (0.050 grams, 0.0001 mole) and N-methyldicyclohexylamine (0.49 grams, 0.0025 mole). This solution was purged for another 10 min with nitrogen, and sealed, stirred vigorously and heated to 90° C. for 48 hours. Dichloromethane (50 mL) and water (100 mL) were added. The organic layer was separated, and the aqueous layer was extracted with dichloromethane (2×50 mL). The combined organic layers were washed with water (2×100 mL) and brine (100 mL), dried over anhydrous magnesium sulfate, and filtered, and then the solvent was completely removed. The residue was passed though a celite plug with diethyl ether as the eluent, the solvent was completely removed. The residue was purified with 50 grams of LH-20 Sephadex size exclusion chromatography using tetrahydrofuran as the eluent to result in [23] (0.350 grams). According to ESI (2095.4, M²⁺+2Na), meaning up to 15 substitutions was achieved. ¹H NMR (300 MHz, CDCl₃, PPM) δ 7.5-6.5 (br, 28H), 5.60-5.59 (m, 1H, intensity was normalized to these peaks), 4.61-4.47 (br, 7H), 4.09-3.41 (br, 49H), 2.10-1.40 (br, 75H).

### Example 9

### Preparation of Compound (24)

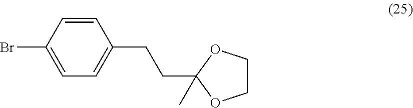

To a 100 mL round bottom flask was added 4-(4-bromophenyl)butan-2-one (5.5 grams, 0.024 moles), ethane diol (4 grams, 0.064 moles), dry toluene (50 mL), p-toluene sulfonic acid (0.13 grams, 0.00068 moles) and fitted to a dean-stark apparatus. The reaction mixture was heated to 130° C. overnight. The solvent was removed under reduced pressure. Dichloromethane (100 mL) and water (100 mL) were added. The organic layer was separated, and the aqueous layer was extracted with dichloromethane (2×50 mL). The combined organic layers were washed with water (2×50 mL) and brine (50 mL), dried over anhydrous magnesium sulfate, filtered, and the solvent was removed to result [25] as a colourless oil (6 grams, ˜91% yield). ¹H NMR (300 MHz, CDCl₃, PPM) δ 7.39-7.36 (m, 2H), 7.07-7.05 (m, 2H), 3.99-3.93 (m, 4H), 2.69-2.63 (m, 2H), 1.95-1.89 (m, 2H), 1.35 (s, 3H).

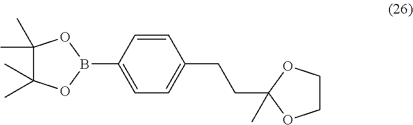

To a 50 mL round bottom flask was added [25] (0.52 grams, 0.0019 moles) and 8 mL of freshly distilled tetrahydrofuran. This solution was cooled to −78° C. and 1.6 M n-BuLi (1.4 mL, 0.0022 mole) was added dropwise. This solution was stirred for 2 hours at −78° C. and 2-isopropoxy-4,4,5,5-tetramethyl-1,3,2-dioxaborolane (0.35 grams, 0.0019 moles) was added. This solution was added to water (100 mL) after gradually allowing this solution to warm to room temperature overnight. The organic layer was separated, and the aqueous layer was extracted with dichloromethane (3×100 mL). The combined organic layers were washed with water (1×100 mL) and brine (100 mL), dried over anhydrous magnesium sulfate, filtered and the solvent was removed under reduced pressure. The residue was purified by silica gel chromatography using a gradient solvent hexane:ethylacetate system to result in a colourless oil containing [26] (0.53 grams, ˜90% yield). ¹H NMR (300.1 MHz, CDCl₃, PPM) δ 7.73-7.71 (m, 2H), 7.21-7.19 (m, 2H), 4.01-3.92 (m, 4H), 2.74-2.70 (m, 2H), 1.97-1.93 (m, 2H), 1.54 (s, 3H), 1.32 (s, 12H).

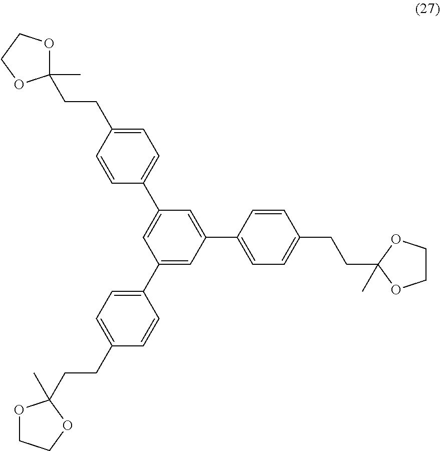

To a 100 mL round bottom flask were added [26] (0.37 grams, 0.0012 moles), tribromobenzene (0.10 grams, 0.00037 moles), toluene (10 mL) and 2M sodium carbonate (1 mL), and the solution was purged with nitrogen gas for 15 min. To this solution was added tetrakis(triphenylphosphine)palladium(0) (0.04 grams, 0.000033 moles) and the solution was purged-degassed with nitrogen three times and reacted under a nitrogen atmosphere for 48 hours. Dichloromethane (25 mL) and water (25 mL) were added. The organic layer was separated, and the aqueous layer was extracted with dichloromethane (2×25 mL). The combined organic layers were washed with water (2×25 mL) and brine (25 mL), dried over anhydrous magnesium sulfate, and filtered, and then the solvent was completely removed. The residue was purified by silica gel chromatography using a gradient solvent dichloromethane:ethyl acetate system, from 0% ethyl acetate resulting in [27] as a colourless oil (0.115 grams, ˜48% yield). ¹H NMR (300.1 MHz, CDCl₃, PPM) δ 7.72 (s, 3H), 7.61-7.58 (m, 6H), 7.31-7.29 (m, 6H), 4.03-3.92 (m, 12H), 2.79-2.75 (m, 6H), 2.03-1.99 (m, 6H), 1.39 (s, 9H).

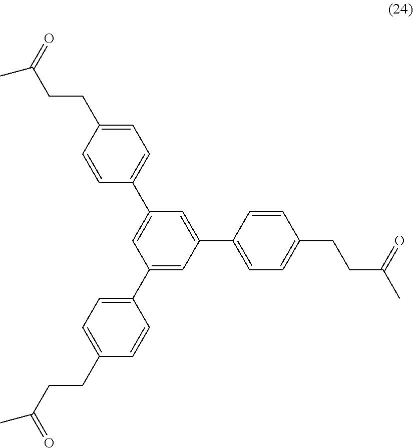

To a 50 mL round bottom flask was added [27] (0.115 g, 0.00018 mole), dioxane (10 mL), 10% HCl solution (10 mL). This solution was heated to 70° C. overnight. The solvent was removed under reduced pressure. Dichloromethane (100 mL) and water (50 mL) were added. The organic layer was separated and dried over anhydrous magnesium sulfate, and filtered, and then the solvent was removed. The residue was purified by silica gel chromatography using a gradient solvent dichloromethane:ethyl acetate system resulting in a colourless oil containing [24] (0.091 grams, ˜quantitative yield). ¹H NMR (300 MHz, CDCl₃, PPM) δ 7.70 (s, 3H), 7.60-7.75 (m, 6H), 7.29-7.26 (m, 6H), 2.97-2.92 (m, 6H), 2.82-2.77 (m, 6H), 2.15 (s, 9H).

### Example 10

### Preparation of Compound (28)

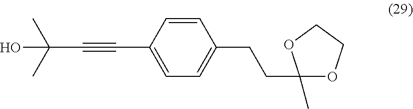

A mixture [25] (2 g, 7.38 mmol) as prepared in Example 9, 2-methyl-3-butyn-2-ol (4.3 mL, 43 mmol) and Et₃N (10 mL, 71.7 mmol) in toluene (80 mL) was purged with Ar for 20 min. Pd(PPh₃)₄ (0.4 g, 0.35 mmol) was then added and the mixture was purged for 2 min, followed by addition of CuI (150 mg, 0.79 mmol). The reaction mixture was purged for another 5 min and then stirred at 45° C. under an atmosphere of Ar and in dark for 6 days. The mixture was cooled to room temperature and filtered through a pad of Celite. The solid residue was rinsed thoroughly with diethyl ether. The combined organic wash was concentrated under reduced pressure and the residue was purified by column chromatography on silica gel using petroleum spirit (40-60° C.) containing increasing quantities of diethyl ether. The desired product [29] was eluted with 25-30% diethyl ether in petroleum spirit (1.60 g, 79%). ¹Hnmr (500 MHz, CDCl₃) δ 7.21, d, 2H, J=8.3 Hz, Ar—H, 7.02, d, 2H, J=8.3 Hz, Ar—H, 3.87, m, 4H, —O—CH₂—CH₂—O—; 3.37, br.s., 1H, —OH; 2.61, m, 2H, Ph-CH₂—CH₂—; 1.86, m, 2H, Ph-CH₂—CH₂—; 1.53, s, 6H, —C(CH₃)₂OH; 1.29, s, 3H, —CH₃. ¹³C nmr (125 MHz, CDCl₃) δ 142.21 (Ar—C); 131.31 (Ar—CH); 127.97 (Ar—CH); 119.93 (Ar—C); 109.37 (acetal C), 93.42 (—C≡C—); 81.60 (—C≡C—); 64.94 (—C(CH₃)₂OH); 64.40 (—O—CH₂—CH₂—O—); 40.34 (Ph-CH₂—CH₂—); 31.28 (—C(CH₃)₂OH); 29.79 (Ph-CH₂—CH₂—); 23.72 (—CH₃).

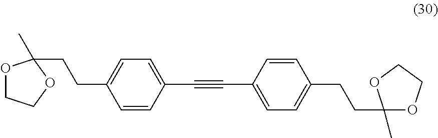

To a 50 mL round bottom flask was added [29] (0.100 grams, 0.00037 moles), [25] (0.100 grams, 0.00037 moles) as prepared in Example 9, toluene (10 mL), triethylamine (1 mL), 5M NaOH (2 mL) and the solution was purged with nitrogen gas for 15 min. To this solution was added tetrakis(triphenylphosphine)palladium(0) (0.020 grams, 0.000002 mole) and CuI (approximately 0.001 grams, 0.0000005 moles). This solution was degassed and purged-degassed with nitrogen three times and reacted at 90° C. under a nitrogen atmosphere for 90 hours. The reaction mixture was quenched with 10% ammonium chloride (100 mL) and dichloromethane (50 mL) was added. The organic layer was separated, and the aqueous layer was extracted with dichloromethane (3×50 mL). The combined organic layers were washed with water (1×50 mL) and brine (50 mL), dried over anhydrous magnesium sulfate, filtered, and then the solvent was removed. The residue was purified using silica gel chromatography (dichloromethane:ethyl acetate gradient) to result in [30] as a colourless oil (0.055 g, yield: ˜37%). ¹H NMR (300 MHz, CDCl₃, PPM) δ=7.43-7.41 (m, 4H), 7.18-7.15 (m, 4H), 4.00-3.94 (m, 8H), 2.74-2.69 (m, 4H), 1.98-1.92 (m, 4H), 1.36 (s, 6H).

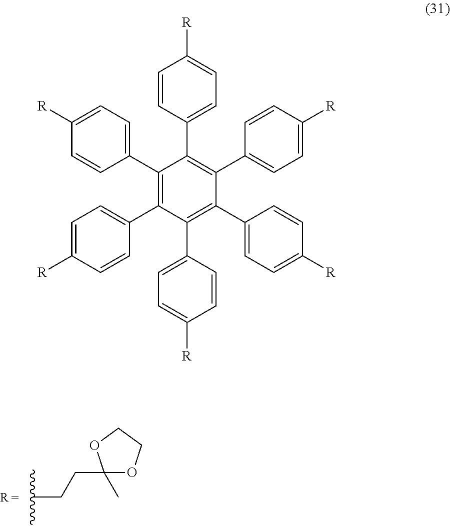

To a 50 mL pressure tube was added [30] (0.022 g, 0.000054 moles) and dioxane (5 mL). This solution was degassed with nitrogen for 15 min and cobalt carbonyl (0.0185 grams, 0.000054 moles) was added. The pressure tube was sealed and heated to 110° C. for 18 hours. The reaction mixture was quenched with water (50 mL), and dichloromethane (50 mL) was added. The organic layer was separated, and the aqueous layer was extracted with dichloromethane (2×50 mL). The combined organic layers were washed with water (1×50 mL) and brine (50 mL), dried over anhydrous magnesium sulfate, filtered, and then the solvent was removed. The residue was purified using silica gel chromatography (dichloromethane:ethyl acetate gradient) to result in [31] a colourless oil (0.055 g, yield: ˜37%). ¹H NMR (300 MHz, CDCl₃, PPM) δ=6.67-6.60 (m, 24H), 3.94-3.82 (m, 24H), 2.47-2.41 (m, 12H), 1.75-1.70 (m, 12H), 1.23 (s, 18H).

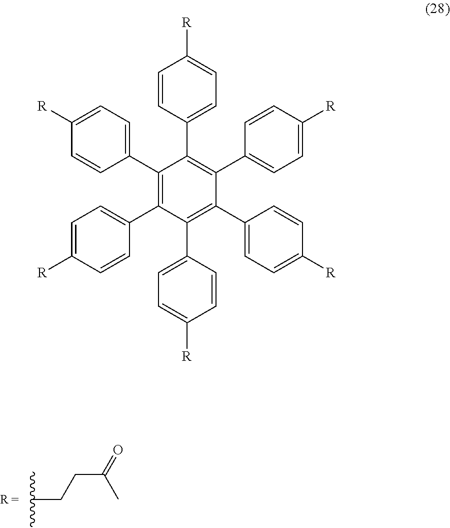

To a 100 mL round bottom flask was added [31] (0.010 g, 0.000008 mole), dioxane (18 mL), 10% HCl solution (10 mL). This solution was heated to 70° C. overnight. The solvent was removed under reduced pressure. Dichloromethane (40 mL) and water (50 mL) were added. The organic layer was separated, and the aqueous layer was extracted with dichloromethane (2×20 mL). The combined organic layers were washed with water (20 mL) and brine (20 mL), dried over anhydrous magnesium sulfate, and filtered, and then the solvent was removed. The residue was purified by silica gel chromatography using a gradient solvent dichloromethane:ethyl acetate system resulting in [28] as a colourless film (0.115 grams, ˜48% yield). ¹H NMR (300 MHz, CDCl₃, PPM) δ 6.67-6.59 (m, 24H), 2.65-2.60 (m, 12H), 2.54-2.48 (m, 12H), 1.94 (s, 18H).

### Example 11

### Preparation of Compound (32)

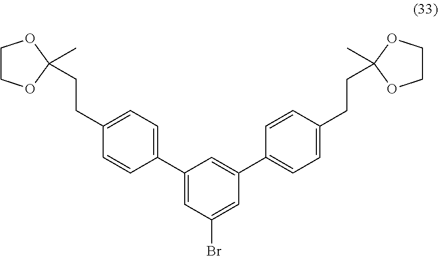

To 100 mL round bottom flask was added [26] (0.8 grams, 0.0025 mole) as prepared in Example 9, 1,3,5-tribromobenzene (0.36 grams, 0.0011 mole), toluene (10 mL) and 2M Na₂CO₃ (3 mL). This solution was purged for 20 min with nitrogen gas. To this solution was added tetrakis(triphenylphosphine)palladium(0) (0.039 grams, 0.000034 moles). The solution was stirred rapidly for 60 hours at 90° C. Dichloromethane (100 mL) and water (100 mL) were added. The organic layer was separated, and the aqueous layer was extracted with dichloromethane (2×100 mL). The combined organic layers were washed with water (2×100 mL) and brine (100 mL), dried over anhydrous magnesium sulfate, and filtered, and then the solvent was completely removed. The residue was purified by silica gel chromatography using a gradient solvent hexane:ethylacetate system resulting in [33] as a colourless oil (0.253 grams, ˜37% yield). ¹H NMR (300 MHz, CDCl₃, PPM) δ 7.67-7.65 (m, 3H), 7.53-7.50 (m, 4H), 7.30-7.27 (m, 4H), 4.04-3.97 (m, 8H), 2.80-2.74 (m, 4H), 2.03-1.97 (m, 4H), 1.38 (s, 6H).

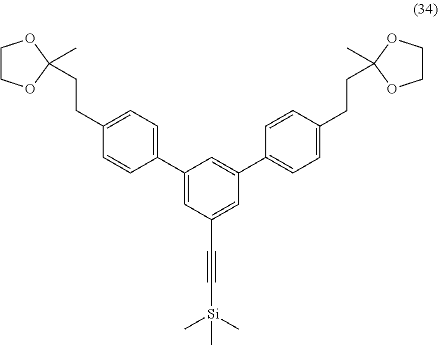

To a 100 mL pressure tube was added [33] (0.075 grams, 0.00019 moles), trimethylsilyl acetylene (1 mL, 0.0117 mole), piperidine (10 mL), toluene (10 mL). This solution was purged for 15 min with nitrogen gas. To this solution was added tetrakis(triphenylphosphine)palladium(0) (0.022 grams, 0.000019 moles) and copper iodide (0.0035 g, 0.000019 moles). This solution was stirred rapidly for 48 hours at 90° C. Dichloromethane (50 mL) and 10% ammonium chloride (50 mL) were added. The organic layer was separated, and the aqueous layer was extracted with dichloromethane (2×25 mL). The combined organic layers were washed with water (50 mL) and brine (50 mL), dried over anhydrous magnesium sulfate, and filtered, and then the solvent was removed. The residue was purified by silica gel chromatography using a gradient solvent dichloromethane: ethylacetate system resulting in [34] as a colourless oil (0.067 grams, ˜87% yield). ¹H NMR (300.1 MHz, CDCl₃, PPM) δ 7.71 (t, j=1.8 Hz, 1H), 7.63 (s, j=1.7 Hz, 2H), 7.55-7.52 (m, 4H), 7.29-7.26 (m, 4H), 4.01-3.96 (m, 8H), 2.79-2.73 (m, 4H), 2.03-1.97 (m, 4H), 1.39 (s, 6H), 0.285 (s, 9H).

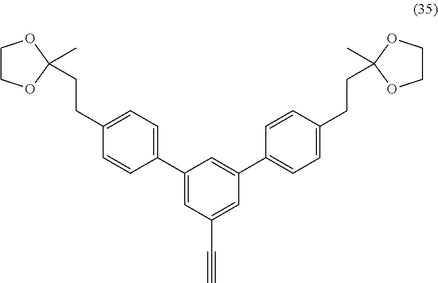

To a 10 mL round bottom flask was added [34] (0.022 g, 0.00004 moles), tetrahydrofuran (2.5 mL) and tetrabutylammonium fluoride (0.011 grams, 0.000043 mole). This solution was stirred at room temperature for 4 hours. The solvent was removed and dichloromethane (25 mL) and water (25 mL) was added. The organic layer was separated, and the aqueous layer was extracted with dichloromethane (2×25 mL). The combined organic layers were washed with water (2×25 mL) and brine (25 mL), dried over anhydrous magnesium sulfate, and filtered, and then the solvent was removed. The residue was purified by silica gel chromatography using a gradient solvent dichloromethane: ethylacetate system to result in [35] as a colourless oil (0.011 grams, ˜c58% yield). ¹H NMR (300.1 MHz, CDCl₃, PPM) δ 7.74 (t, j=1.7 Hz, 1H), 7.66 (d, j=1.8 Hz, 2H), 7.55-7.52 (m, 4H), 7.30-7.27 (m, 4H), 4.01-3.97 (m, 8H), 3.10 (s, 1H), 2.79-2.74 (m, 4H), 2.03-1.97 (m, 4H), 1.39 (s, 6H).

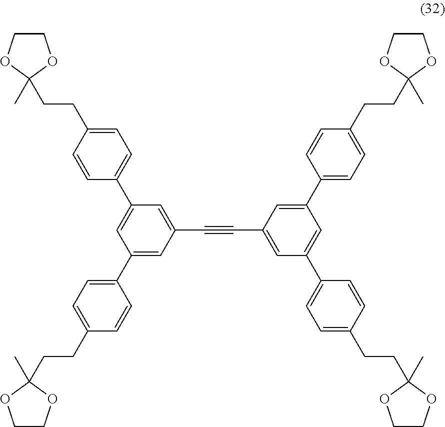

To a 100 mL pressure tube [35] (0.011 grams, 0.000023 moles), [33] (0.013 grams, 0.000023 moles), toluene (5 mL) and piperidine (2 mL) were added. This solution was purged for 20 min with nitrogen gas. To this solution was added tetrakis(triphenylphosphine)palladium(0) (0.005 grams, 0.000005 moles) and copper iodide (0.0004 g, 0.000005 moles). This solution was stirred rapidly for 84 hours at 90° C. Dichloromethane (50 mL) and 10% ammonium chloride (25 mL) were added. The organic layer was separated, and the aqueous layer was extracted with dichloromethane (3×25 mL). The combined organic layers were washed with water (25 mL) and brine (25 mL), dried over anhydrous magnesium sulfate, and filtered, and then the solvent was removed. The residue was purified by silica gel chromatography using a gradient solvent dichloromethane: ethylacetate system resulting in a film containing [32] (0.011 grams, ˜52% yield). ¹H NMR (300.1 MHz, CDCl₃, PPM) δ 7.78-7.75 (m, 6H), 7.62-7.60 (m, 8H), 7.34-7.32 (m, 8H), 4.07-3.98 (m, 16H), 2.83-2.79 (m, 8H), 2.07-2.02 (m, 8H), 1.43 (s, 12H).

### Example 12

### Preparation of Compound (36)

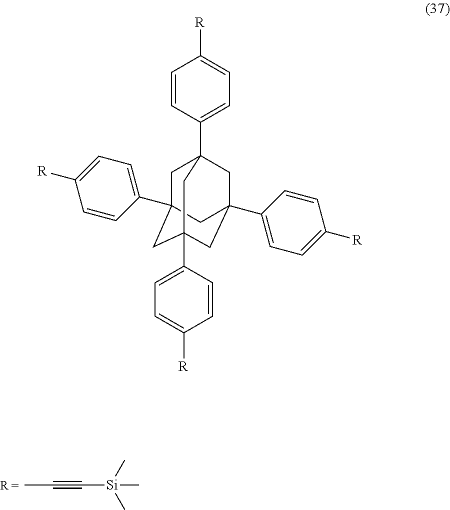

Tetrakis(4-iodophenyl)-1,1′,1″,1′″-adamantane (48.2 mg, 0.05 mmol) was dissolved in dry toluene (10 mL) in a sealed pressure tube, under anhydrous conditions. To this, triethylamine (0.30 mL, 2.15 mmol) was added and the reaction vessel purged with Ar (g) for 4 min before adding trimethylsilylacetylene (0.30 mL, 2.17 mmol). The reaction was purged for another minute before adding Pd(PPh₃)₄ (12.2 mg, 0.01 mmol) and CuI (6.3 mg, 0.03 mmol) and left to stir at 45° C. for 5 days, protected from light. Catalyst was removed by filtering through a celite plug, rinsed with toluene. Solvent was removed in vacuo and crude compound purified using flash silica column chromatography (SiO₂; 0% diethyl ether/petroleum spirit to 1%, gradient elution) afforded [37] (42.2 mg, in quantitative yields) as a white solid; ¹H NMR (400 MHz; CDCl₃) δ 7.42, 2.10, 0.27; ¹³C NMR (400 MHz; CDCl₃) δ 149.4, 132.1, 124.9, 121.1, 105.1, 94.0, 46.7, 39.3, 1.1, 0.0.

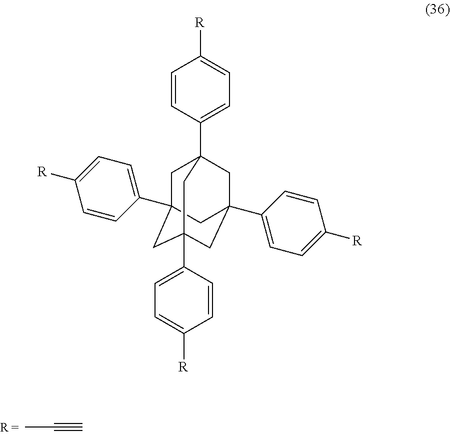

[37] (42.2 mg, 0.05 mmol) was dissolved in dry THF (5 mL), under anhydrous conditions before n-Bu₄N⁺F⁻ (0.3 mL, 0.30 mmol) was added and stirred at RT overnight, protected from light. Solvent was removed in vacuo to give a yellow oil that was dissolved in EtOAc, washed with 10% HCl (3×20 mL), saturated sodium bicarbonate (1×20 mL), dried (MgSO₄), filtered, and the solvent removed in vacuo to give [36] as a white solid. (32.3 mg, quantitative yields). ¹H NMR (500 MHz; CDCl₃) 7.49, 7.48, 7.43, 7.41, 3.06, 2.12.

### Example 13

### Preparation of Compound (38)

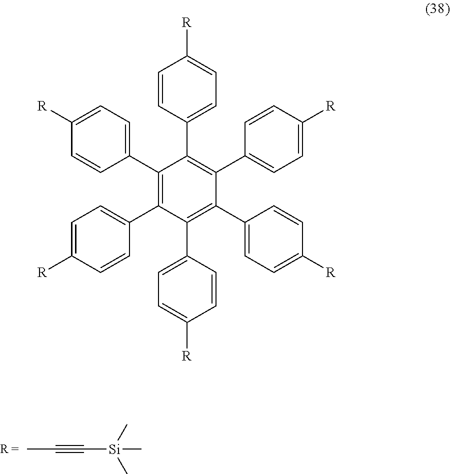

Hexakis(4-iodophenyl)benzene (48.2 mg, 0.04 mmol) was dissolved in dry toluene (5 mL) in a sealed pressure tube, under anhydrous conditions. To this, triethylamine (0.32 mL, 2.24 mmol) was added and the reaction vessel purged with Ar (g) for 15 min before adding trimethylsilylacetylene (0.19 mL, 1.35 mmol). The reaction was purged for another minute before adding Pd(PPh₃)₄ (11 mg, 4.4% neq×6) and CuI (2.1 mg, 5% neq×6) and left to stir at 90° C. for 8 days, protected from light. Catalyst was removed by filtering through a celite plug, rinsed with toluene. Solvent was removed in vacuo and crude compound purified using flash silica column chromatography (SiO₂; 0% diethyl ether/petroleum spirit to 3%, gradient elution) afforded (38) (37.1 mg, 90%) as a white solid; ¹H NMR (500 MHz; CDCl₃) δ 6.96, 6.65, 0.18; ¹³C NMR (500 MHz; CDCl₃) δ 140.1, 139.8, 130.9, 130.8, 120.3, 105.2, 94.1, −0.1.

### Example 14

### Preparation of Compound (39)

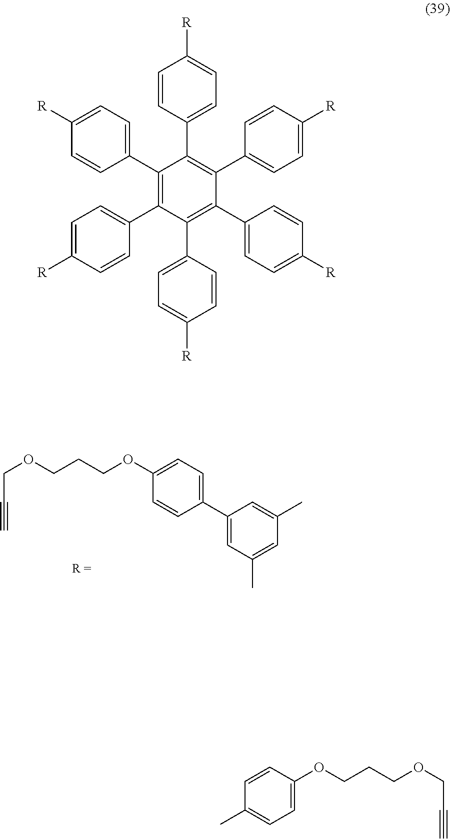

A solution of [13] (9.60 mg, 0.0025 mmol) as prepared in Example 3, in MeOH/THF (1:1, 2 mL) and 4 drops of HCl was stirred at room temperature for 2 days, protected from light. Solvent was removed in vacuo and a white residue remained. This hydroxylated intermediate was dissolved in dry DMF (3 mL) under anhydrous conditions, and cooled in an ice bath to 0° C. Sodium hydride (7.8 mg, 0.195 mmol) was added to the reaction vessel and stirred for 2.5 hours. After this time, propargyl bromide (92 mg, 0.62 mmol) was added and the reaction was left to warm to room temperature and stirred for 3 days. Remaining NaH was quenched with isopropanol (4 drops) before the solvent was evaporated in vacuo. Purification to remove NaH dispersion oil was achieved by sonication of the product with petroleum ether (4×2 mL) which was decanted out, to give (39) as an amber solid (10.3 mg, 94%) ¹H NMR (500 MHz, CDCl₃) δ 7.55 (2H, Ar—H), 7.49 (211, Ar—H), 7.31 (214, Ar—H), 7.05 (2H, Ar—H), 6.87 (3H, Ar—H), 4.15 (d, J_{CH2, CH} 2.55 Hz, 2H, Propargyl-CH₂—), 4.04 (m, 2H, Propyl —CH₂), 3.70 (m, 2H, Propyl —CH₂), 2.41 (s, 1H, CH), 2.06 (m, 2H, Propyl —CH₂—).

### Example 15

### Preparation of Compound (40)

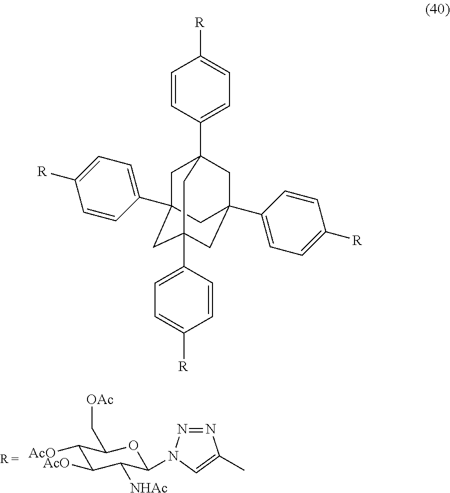

2-Acetamido-2-Deoxy-3,4,6-Tri-O-Acetyl-β-D-Glucopyranosyl Azide (26.4 mg, 0.07 mmol) [Cunha et al. Nucleosides, Nucleotides & Nucleic Acids, 2001, 20(8), 1555-1569; Macmillan et al., Organic Letters, 2002, 4(9), 1467-1470] and [36] (7.1 mg, 0.01 mmol) as prepared in Example 12, were stirred in dry toluene (2 mL)/dry DMF (0.2 mL) in a schlenk tube, under anhydrous conditions. To this, pentamethyldiethylene triamine (PMDETA) (12 μL, 0.06 mmol) was added and the reaction vessel purged with N₂ (g) for 15 min. Cu(I) Br (7.1 mg, 0.05 mmol) was added and the reaction purged for a further 2 min before being left to stir at room temperature for 4 days, protected from light. Air was bubbled slowly through the lime coloured solution and the resultant solid Cu(II) formed filtered off (rinsing with DCM) in a celite plug. Solvent was removed in vacuo to give a dark green solid. Purification using flash silica pipette sized column chromatography (SiO₂; 20% ethyl acetate/hexane to 100%, gradient elution) afforded [40] (15.6 mg) as a white solid. Recrystallization using EtOH/DCM afforded 12.7 mg of a white solid. LRESIMS m/z 1035 [M+2Na]²⁺ or [2M+4Na]⁴⁺.

### Example 16

### Synthesis of Peptides of SEQ ID NO. 4, 5, 6, 7 and 8

The peptides, H-CVKLT-NH₂ (SEQ ID NO. 4), H-VGKAMY-NH₂ (SEQ ID NO. 5) and H—CPKEFKQ₁-NH₂ (SEQ ID NO. 6), AOAA-CVKLT-NH₂ (SEQ ID NO. 7) and AOAA-VGKAMY-NH₂ (SEQ ID NO. 8) [Enshell-Seijfters et al., J. Mol. Biol., 2003, 334, 87-101] were synthesised using Rink amide MBHA resin (1 g, 0.72 mmol/g) and HBTU as coupling reagent under standard Fmoc solid phase peptide synthesis protocols. The resin-bound peptides were stirred with a mixture of TFA/TIPS/H₂O (9.5:0.25:0.25, 10 mL) for 3 h at room temperature, purified by rp-HPLC, and analysed by analytical rp-HPLC and mass spectrometry. Analytical rp-HPLC was performed on a Phenomenex Jupiter 3μ C18 250×4 mm column, using gradient mixtures of water/0.1% TFA (solvent system A) and water 10%/acetonitrile 90%/TFA 0.1% (solvent system B).

H-CVKLT-NH₂ (SEQ ID NO: 4): ESIMS: M+H=562.3; rp-HPLC: R(t)=16.13 min.

H-VGKAMY-NH₂ (SEQ ID NO: 5): ESIMS: M+H=667.4; rp-HPLC: R(t)=16.21 min.

H-CPKEFKQI-NH₂ (SEQ ID NO: 6): ESIMS: M+H=991.5; rp-HPLC: R(t)=17.47 min.

AOAA-CVKLT-NH₂ (SEQ ID NO: 7) and AOAA-VGKAMY-NH₂ (SEQ ID NO: 8) were obtained by coupling aminooxyacetic acid to resin-bound peptides SEQ ID NO: 4 and SEQ ID NO: 5 respectively, using HBTU and DIPEA in DMF, followed treatment with a mixture of TFA/TIPS/H₂O (9.5:0.25:0.25, 10 mL) for 3 h at room temperature and purification by rp-HPLC.

AOAA-CVKLT-NH₂ (SEQ ID NO: 7): ESIMS: M+H=635.3.

AOAA-VGKAMY-NH₂ (SEQ ID NO: 8): ESIMS: M+H=740.4.

### Example 17

### Preparation of Protected Pepetides H—C(Trt)VK(Boc)LT(tBu)-NH₂ (SEQ ID NO: 9) and II-VGK(Boc)AMY(tBu)-NH₂ (SEQ ID NO: 10)

Fmoc-Thr(tBu)-OH and Fmoc-Tyr(tBu)-OH required for the synthesis of (SEQ ID NO: 9) and (SEQ ID NO: 10), respectively were converted to Fmoc-Thr(tBu)-NH₂ and Fmoc-Tyr(tBu)-NH₂ as described by Singh et. al., J. Am. Chem. Soc., 2001, 123, 333-334. The peptides were then synthesized by standard Fmoc solution phase peptide synthesis protocols in 0.5 mmol scale using BOP coupling reagent in the presence of DIPEA as base and DMF as solvent. DBU in DCM as solvent was used to remove the Fmoc protecting group.

H—C(Trt)VK(Boc)LT(tBu)-NH₂ (SEQ ID NO: 9): ESIMS: M+H=960.6.

H-VGK(Boc)AMY(tBu)-NH₂ (SEQ ID NO: 10): ESIMS: M+H=823.5.

### Example 18

### Synthesis of Protected Peptides H-VGK(Boc)AMY(tBu)-OMe (SEQ ID NO: 11) and H—C(Trt)PK(Boc)E(OtBu)FK(Boc)Q(NHTrt)I—OBn (SEQ ID NO: 12)

These peptides were synthesised using 2-chlorotrityl chloride resin (˜1.7 g, 1.01 mmol/g), HBTU as coupling reagent, 3 mole equivalents of amino acid and DIPEA in DMF (30 mL) under standard Fmoc solid phase peptide synthesis protocols and cleaved off the resin with 1% TFA/DCM (20 mL×20 min x 3), [Singh et al., J. Am. Chem. Soc., 2005, 127, 6563-6572]. For SEQ ID NO: 11, the carboxylic acid end group was converted to methyl ester using MeI and DIPEA in DCM as solvent and for SEQ ID NO: 12 the carboxylic acid end group was converted to benzyl ester using benzyl bromide and DIPEA in DMF as solvent. The N-terminus Fmoc protecting group was removed with DBU in DCM.

H-VGK(Boc)AMY(tBu)-0Me (SEQ ID NO: 11): ESIMS: M+H=838.5; rp-HPLC: R(t)=27.51 min.

H—C(Trt)PK(Boc)E(OtBu)FK(Boc)Q(NHTrt)I—OBn (SEQ ID NO: 12): ESIMS: (M+2H)/2=912.1, M+H=1823.2.

### Example 19

### Synthesis of Peptides Conjugates via Peptide Bond Formation

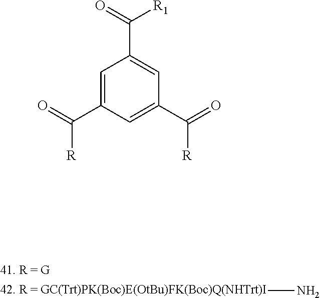

Three copies of resin-bound protected C(Trt)PK(Boc)E(OtBu)FK(Boc)Q(NHTrt)I—NH₂ (SEQ ID NO: 13, 162 mg) was coupled to compound (41) (Ashton et al. Chem. Eur. J., 1996, 2, 1115-1128) (5 mg, 1.31×10−5 mol) using EDC (14 mg, 7.3×10⁻⁵ mol) and HOBT (10 mg, 7.4×10⁻⁵ mol) in the presence of benzene (0.5 mL) and DMF (4 mL) as described by Singh et. al., (Abstract, 231 ACS National Meeting, Atlanta, Ga., USA, Mar. 26-30, 2006). The resin was then washed thoroughly, dried and stirred with a mixture of TFA/TIPS/H₂O (9.5:0.25:0.25, 2 mL) for 3 h at room temperature. The reaction mixture was concentrated and the residue was purified by rp-HPLC to give the conjugate (42) as a white solid (31 mg, 73%). ESIMS: (M+3H)/3=1100.3, (M+4H)/4=825.2, (M+5H)/5=660.3.

### Example 20

### Synthesis of Compounds with Three Different Peptides

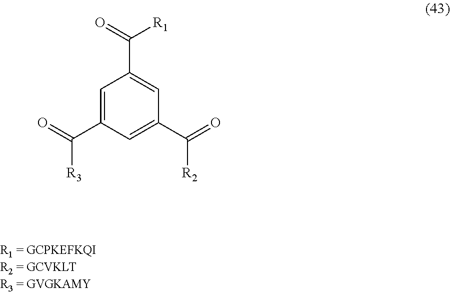

Coupling of a copy of resin-bound protected C(Trt)PK(Boc)E(OtBu)FK(Boc)Q(NHTrt)I—NH₂ (SEQ ID NO: 13) to scaffold (41) as described by Singh et. al. J. Am. Chem. Soc., 2001, 123, 333-334, followed by coupling of the side-chain protected H—C(Trt)VK(Boc)LT(tBu)-NH₂ (SEQ ID NO: 9) and H-VGK(Boc)AMY(tBu)-NH₂ (SEQ ID NO: 10) in one mole equivalent portions, using EDC and HOBT in a mixture of DMF and benzene as solvent. Then treatment of the product with a mixture of TFA/TIPS/H₂O (9.5:0.25:0.25) for 3 h at room temperature to provide Compound (43).

### Example 21

### Synthesis of Compounds with different peptides

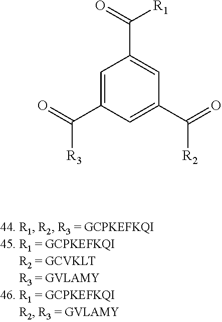

One equivalent of resin-bound peptide C(Tr)PK(Boc)E(OtBu)FK(Boc)Q(NHTrt)I—NH₂ (SEQ ID NO: 13, 142 mg) was shaken with compound (41, 12.1 mg, 3.17×10⁻⁵ mol), EDC (23 mg, 12.0×10⁻⁵ mol) and HOBT (16 mg, 11.8×10⁻⁵ mol) in the presence of benzene (0.5 mL) and DMF (4 mL) for 48 h. The resin was then washed with DMF. Protected peptides (SEQ ID NO: 9, 30 mg, 3.12×10⁻⁵ mol) was then added to the resin and shaken in the presence of EDC (23 mg, 12.0×10⁻⁵ mol), HOBT (16 mg, 11.8×10⁻⁵ mol), benzene (0.5 mL) and DMF (4 mL) for 72 h. After washing the resin the peptide (SEQ ID NO: 10, 26 mg, 3.15×10⁻⁵ mol) was added and shaken in the presence of EDC (23 mg, 12.0×10⁻⁵ mol), HOBT (16 mg, 11.8×10⁻⁵ mol), benzene (0.5 mL) and DMF (4 mL) for 48 h. The resin was then washed thoroughly with DMF followed by MeOH and DCM, dried and stirred with a mixture of TFA/TIPS/H₂O (9.5:0.25:0.25, 4 mL) for 3 h at room temperature. The reaction mixture was concentrated and the residue was purified by rp-HPLC and analysed by mass spectrometry. Conjugate (44) was the major product together with traces of conjugates (45) and (46).

Conjugate 44. ESIMS: (M+3H)/3=1100.3, (M+4H)/4=825.2, (M+5H)/5=660.3.

Conjugate 45. ESIMS: (M+H+3Na)/4=653.8.

Conjugate 46. ESIMS: (M+4H+Na)/5=535.4.

### Example 22

### Synthesis of Compounds with Three Different Peptides

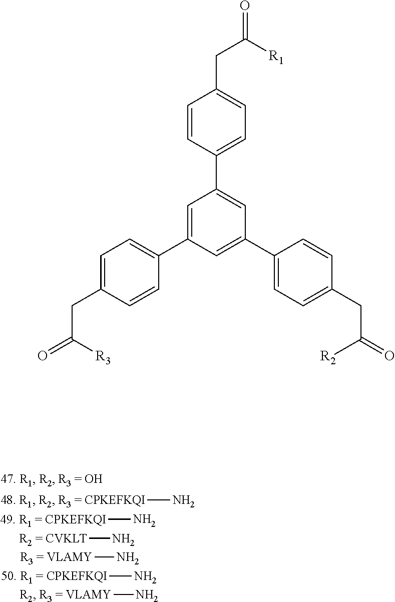

One equivalent of resin-bound protected peptide C(Trt)PK(Boc)E(OtBu)FK(Boc)Q(NHTrt)I—NH₂ (SEQ ID NO: 13, 100 mg) was shaken with compound (47, 14.7 mg, 3.05×10⁻⁵ mol), EDC (22 mg, 11.5×10⁻⁵ mol) and HOBT (18 mg, 13.3×10⁻⁵ mol) in the presence of benzene (0.6 mL) and DMF (5 mL) for 48 h. The resin was then washed with DMF. Protected peptides (SEQ ID NO: 9, 29.3 mg, 3.05×10⁻⁵ mol) was then added to the resin and shaken in the presence of EDC (23 mg, 12.0×10⁻⁵ mol), HOBT (18 mg, 13.38×10⁻⁵ mol), benzene (0.6 mL) and DMF (5 mL) for 72 h. After washing the resin the protected peptide (SEQ ID NO: 10, 25 mg, 3.03×10⁻⁵ mol) was added and shaken in the presence of EDC (23 mg, 12.0×10⁻⁵ mol), HOBT (16 mg, 11.8×10⁻⁵ mol), benzene (0.6 mL) and DMF (5 mL) 48 h. The resin was then washed thoroughly with DMF followed by MeOH and DCM, dried and stirred with a mixture of TFA/TIPS/H₂O (9.5:0.25:0.25, 4 mL) for 3 h at room temperature. The reaction mixture was concentrated and the residue was purified by rp-HPLC and analysed by mass spectrometry. Conjugate (48) was the major product together with traces of conjugates (49) and (50).

Conjugate 48. ESIMS: (M+3H)/3=1134.1, (M+4H)/4=850.5.

### Example 23

### Synthesis of Compound with Three Different Peptides

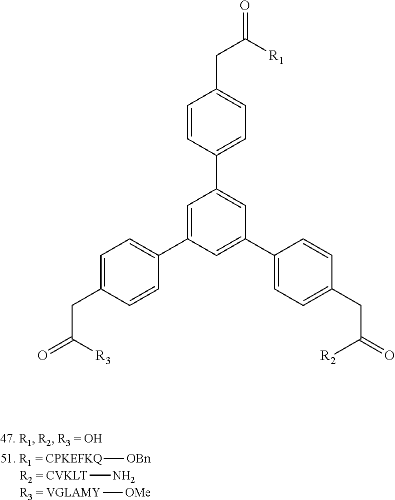

A mixture of compound (47, 20 mg, 4.16×10⁻⁵ mol) and BOP (110 mg, 2.5×10⁻⁴ mol) in DMF (100 mL) and benzene (5 mL) was stirred with DIPEA (0.14 mL, 8.04×10⁻⁴ mol) for 10 min. Side-chain protected peptide (SEQ ID NO: 9, 40 mg, 4.16×10⁻⁵ mol) was added and stirred for 24 h under an atmosphere of Argon at room temperature. Peptide

(SEQ ID NO: 11, 35 mg, 4.18×10⁻⁵ mol) was then added to the reaction mixture and stirred for further 24 h. After that peptide (SEQ ID NO: 12, 76 mg, 4.18×10⁻⁵ mol) was added to the mixture and stirred for further 48 h. A sample (10 mL) was taken and concentrated under vacuum at room temperature. The residue was stirred with a mixture of TFA/TIPS/H₂O (9.5:0.25:0.25, 2 mL) for 3 h at room temperature, concentrated and analysed by mass spectrometry. Conjugated (51) was detected as the major product.

ESIMS: (M+3H)/3=917.7, (M+4H)/4=685.5, (M+5H)/5=551.1.

1. A compound having formula (I):
Scaffold-[L-(Antigen)ₜ]_{y} (I)
wherein Antigen represents at least a portion of a target antigen for modulating an immune response;
wherein t is 0 or an integer of at least 1;
wherein y is at least 1;
wherein the number of Antigens on the Scaffold is at least 2;
wherein L is a linking group or a covalent bond, wherein when L is a covalent bond, the covalent bond is a single bond attached to a sp or sp² hybridized atom of the Scaffold and when L is a linking group, the linking group is attached to the Scaffold through a single bond attached to a sp or sp² hybridized atom; whereby the Scaffold is sufficiently rigid to maintain the relative position of the single bonds attached to sp or sp² hybridized atoms.

2. A compound of formula (I) according to claim 1 having formula (II):
Scaffold[L-(Antigen)ₜ]_{y} (II)
wherein the Scaffold comprises a group
Core-[Spacer]_{z}
wherein the Core is a central atom or group and the spacer is a group wherein each Spacer, alone or in combination with the Core, comprises at least one unbranched or branched moiety comprising at least one group selected from aryl, heteroaryl, alkenyl, acetylenyl and carbonyl,
wherein the number of Antigens on the Scaffold is at least 2; and
wherein z is at least 1.

3. A compound of formula (I) according to claim 1 having formula (III):
Scaffold-[L-(Antigen)ₜ]_{y} (III)
wherein the Scaffold comprises a group
Core-[Spacer]_{z}
wherein the Core is a central atom or group and the Spacer is a group wherein each Spacer, alone or in combination with the Core, comprises at least one partially conjugated unbranched or branched moiety comprising at least two groups selected from aryl, heteroaryl, alkenyl, acetylenyl and carbonyl,
wherein the number of Antigens on the Scaffold is at least 2; and
wherein z is at least 1.

4. A compound according to claim 1 wherein the Scaffold comprises an acetylene group, an aryl group, a caged hydrocarbon group or a silsesquioxane group.

5. A compound according to claim 2 wherein the Spacer is an unbranched moiety.

6. A compound according to claim 2 wherein the Spacer is a branched moiety.

7. A compound according to claim 2 wherein each Spacer bears one antigen, at least one Spacer bears more than one antigen, or each Spacer bears more than one antigen.

8-9. (canceled)

10. A compound according to claim 1 wherein L is a covalent bond or a linking group comprising a carboxylic acid, an amine, an oxime, a heteroaryl group, an amino acid, a dipeptide or tripeptide.

11. A compound according to claim 1 wherein the target antigen stimulates or enhances an immune response to a viral infection or a bacterial infection.

12. A compound according to claim 11 wherein the viral infection is HIV.

13. A compound according to claim 11 wherein the bacterial infection is caused by Staphylococcus spp.

14. A compound according to claim 1 which is a compound of formula (V): 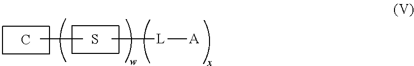
wherein C is an aryl or heteroaryl group, a caged hydrocarbon group, a caged silicon containing group, an acetylenyl group or a vinyl,
S is selected from aryl, heteroaryl, acetylenyl, alkenyl or carbonyl or a group: 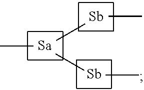
Sa is selected from aryl, heteroaryl, acetylenyl or carbonyl;
Sb is selected from aryl, heteroaryl, acetylenyl or carbonyl or is a group: 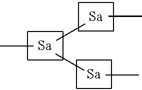
L is a covalent bond or a linking group;
A represents at least a portion of a target antigen for modulating an immune response;
w is an integer from 1 to 6; and
x is at least 2.

15. A compound of formula (I) according to claim 1 having the formula (IV):
CORE-[DENDRITE]n (IV)
wherein CORE represents an atom or group, n represents an integer of at least 1, and DENDRITE, which may be the same or different if n is greater than 1, represents an at least partly conjugated dendritic molecular structure comprising groups selected from aryl, heteroaryl, alkenyl, acetylenyl and carbonyl, and said DENDRITE comprising at least one antigen; and wherein said CORE terminating at a bond to a sp² hybridized atom which forms part of a moiety that has at least two substituents.

16. A compound according to claim 15 wherein a compound of formula (VI): 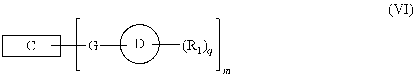
wherein C is an aryl or heteroaryl group;
G is absent is selected from the group consisting of —C(O)—, —CR₂═CR₃— or —C≡C—;
each R₁ is independently selected from 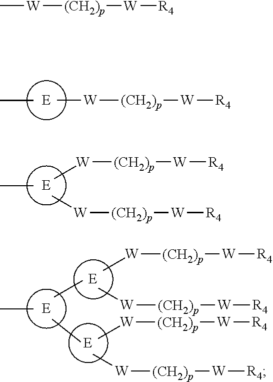
R₂ is hydrogen, alkyl or a polar group,
R₃ is hydrogen, alkyl, a polar group or 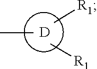
D is an aryl or heteroaryl group;
each E is independently selected from an aryl or heteroaryl group;
each R₄ is the same or different and is an antigen and its attachment to D or E;
each W is independently absent or is independently selected from —O—, —NH—, —S—, —S(O)— or S(O)₂;
each p is the same or different and is 0 or an integer from 1 to 10;
q is an integer of at least 2; and
m is an integer from 1 to 10.

17. An immunomodulating composition, which comprises a compound according to claim 1 and optionally a pharmaceutically acceptable carrier, diluent or adjuvant.

18. A method for modulating an immune response in a subject, the method comprising administering to the subject a compound according to claim 1, and optionally a pharmaceutically acceptable carrier, diluent or adjuvant.

19. A method according to claim 18 wherein the method comprises the treatment or prevention of a disease or condition.

20. A method according to claim 19 wherein the or each antigen of the compound corresponds to at least a portion of a corresponding target antigen associated with the disease or condition, and stimulates or enhances an immune response to that target antigen.

21. A method according to claim 19 wherein each antigen of the compound corresponds to at least a portion of a corresponding target antigen associated with the disease or condition, and attenuates or otherwise suppresses or reduces an immune response or elicits a tolerogenic response to that target antigen.

22-23. (canceled)

24. A compound according to claim 3 wherein the Spacer is an unbranched moiety.

25. A compound according to claim 3 wherein the Spacer is a branched moiety.

26. A compound according to claim 3 wherein each Spacer bears one antigen, at least one Spacer bears more than one antigen, or each Spacer bears more than one antigen.
